SEQUENCE LISTING (5 sequences)

SEQ ID NO: 1 - protein, 1299 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 2 - protein, 1299 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGRQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 3 - protein, 1299 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGRTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - DNA, 3903 bp, synthetic construct
  source 1..3903: mol_type = other DNA
atgagcatctatcaggagttcgtgaataagtacagcctgtccaagaccctgcggtttgagctgatcccccagggcaagacactggagaacatcaaggccaggggcctgatcctggacgatgagaagcgcgccaaggactataagaaggccaagcagatcatcgataagtaccaccagttctttatcgaggagatcctgagcagcgtgtgcatctctgaggatctgctgcagaattacagcgacgtgtatttcaagctgaagaagtctgacgatgacaacctgcagaaggacttcaagagcgccaaggacaccatcaagaagcagatcagcgagtatatcaaggactccgagaagtttaagaatctgttcaaccagaatctgatcgatgccaagaagggccaggagtccgacctgatcctgtggctgaagcagtctaaggacaatggcatcgagctgttcaaggccaactctgatatcaccgatatcgacgaggccctggagatcatcaagagctttaagggctggaccacatactttaagggcttccacgagaacaggaagaacgtgtacagcagcaacgacatccctacaagcatcatctaccgcatcgtggatgacaatctgccaaagttcctggagaacaaggccaagtatgagtccctgaaggacaaggcccccgaggccatcaattacgagcagatcaagaaggatctggccgaggagctgaccttcgatatcgactataagacatccgaggtgaaccagcgggtgttttctctggacgaggtgtttgagatcgccaatttcaacaattacctgaaccagtccggcatcaccaagttcaatacaatcatcggcggcaagtttgtgaacggcgagaataccaagagaaagggcatcaacgagtacatcaatctgtatagccagcagatcaacgacaagaccctgaagaagtacaagatgagcgtgctgttcaagcagatcctgtccgatacagagtctaagagctttgtgatcgataagctggaggatgactctgacgtggtgaccacaatgcagagcttttatgagcagatcgccgccttcaagaccgtggaggagaagtctatcaaggagacactgagcctgctgttcgatgacctgaaggcccagaagctggacctgtctaagatctacttcaagaacgataagtccctgaccgacctgtctcagcaggtgtttgatgactatagcgtgatcggcaccgccgtgctggagtacatcacacagcagatcgccccaaagaacctggataatccctctaagaaggagcaggagctgatcgccaagaagaccgagaaggccaagtatctgagcctggagacaatcaagctggccctggaggagttcaataagcaccgggatatcgacaagcagtgcagatttgaggagatcctggccaacttcgccgccatccccatgatctttgatgagatcgcccagaacaaggacaatctggcccagatctccatcaagtaccagaaccagggcaagaaggacctgctgcaggcctctgccgaggatgacgtgaaggccatcaaggatctgctggaccagaccaacaatctgctgcacaagctgaagatcttccacatctcccagtctgaggataaggccaatatcctggataaggacgagcacttttatctggtgttcgaggagtgttacttcgagctggccaacatcgtgcccctgtacaacaagatcagaaattatatcacacagaagccttactccgacgagaagtttaagctgaacttcgagaacagcaccctggccaacggctgggataagaataaggagcctgacaacacagccatcctgttcatcaaggatgacaagtactatctgggcgtgatgaataagaagaacaataagatcttcgatgacaaggccatcaaggagaacaagggcgagggctacaagaagatcgtgtataagctgctgcccggcgccaataagatgctgcctaaggtgttcttttccgccaagtctatcaagttctacaacccatccgaggacatcctgcggatcagaaatcactccacccacacaaagaacggctctccccagaagggctatgagaagtttgagttcaatatcgaggattgccggaagtttatcgacttctacaagcagagcatctccaagcaccctgagtggaaggattttggcttcaggtttagcgacacccagcggtacaactccatcgacgagttctacagagaggtggagaatcagggctataagctgacatttgagaacatctctgagagctacatcgacagcgtggtgaatcagggcaagctgtacctgttccagatctataacaaggacttcagcgcctattccaagggccggccaaacctgcacaccctgtactggaaggccctgttcgatgagagaaatctgcaggacgtggtgtataagctgaacggcgaggccgagctgttttacaggaagcagtccatccctaagaagatcacacacccagccaaggaggccatcgccaacaagaataaggacaatcctaagaaggagagcgtgttcgagtacgatctgatcaaggacaagcggttcaccgaggataagttctttttccactgtccaatcacaatcaacttcaagtcctctggcgccaacaagtttaatgacgagatcaatctgctgctgaaggagaaggccaacgatgtgcacatcctgagcatcgaccggggcgagagacacctggcctactataccctggtggatggcaagggcaatatcatcaagcaggataccttcaacatcatcggcaatgacaggatgaagacaaactaccacgataagctggccgccatcgagaaggatagggactccgcccgcaaggactggaagaagatcaacaatatcaaggagatgaaggagggctatctgtctcaggtggtgcacgagatcgccaagctggtcatcgagtacaatgccatcgtggtgttcgaggatctgaacttcggctttaagaggggccgctttaaggtggagaagcaggtgtatcagaagctggagaagatgctgatcgagaagctgaattacctggtgtttaaggataacgagttcgacaagaccggaggcgtgctgagggcataccagctgaccgccccctttgagacattcaagaagatgggcaggcagacaggcatcatctactatgtgccagccggcttcacctccaagatctgccccgtgacaggctttgtgaaccagctgtaccctaagtatgagtccgtgtctaagagccaggagtttttcagcaagttcgataagatctgttataatctggacaagggctacttcgagttttccttcgattataagaactttggcgacaaggccgccaagggcaagtggaccatcgcctctttcggcagccggctgatcaactttagaaattccgataagaaccacaattgggacacccgggaggtgtacccaacaaaggagctggagaagctgctgaaggactacagcatcgagtatggccacggcgagtgcatcaaggccgccatctgtggcgagagcgataagaagtttttcgccaagctgacctccgtgctgaatacaatcctgcagatgcggaacagcaagaccggcacagagctggactacctgatctcccccgtggccgatgtgaacggcaacttcttcgacagcagacaggcccccaagaatatgcctcaggatgccgacgccaacggcgcctatcacatcggcctgaagggcctgatgctgctgggcaggatcaagaacaatcaggagggcaagaagctgaacctggtcatcaagaacgaggagtactttgagttcgtgcagaaccgcaacaattga

SEQ ID NO: 5 - DNA, 3903 bp, synthetic construct
  source 1..3903: mol_type = other DNA
atgagcatctatcaggagttcgtgaataagtacagcctgtccaagaccctgcggtttgagctgatcccccagggcaagacactggagaacatcaaggccaggggcctgatcctggacgatgagaagcgcgccaaggactataagaaggccaagcagatcatcgataagtaccaccagttctttatcgaggagatcctgagcagcgtgtgcatctctgaggatctgctgcagaattacagcgacgtgtatttcaagctgaagaagtctgacgatgacaacctgcagaaggacttcaagagcgccaaggacaccatcaagaagcagatcagcgagtatatcaaggactccgagaagtttaagaatctgttcaaccagaatctgatcgatgccaagaagggccaggagtccgacctgatcctgtggctgaagcagtctaaggacaatggcatcgagctgttcaaggccaactctgatatcaccgatatcgacgaggccctggagatcatcaagagctttaagggctggaccacatactttaagggcttccacgagaacaggaagaacgtgtacagcagcaacgacatccctacaagcatcatctaccgcatcgtggatgacaatctgccaaagttcctggagaacaaggccaagtatgagtccctgaaggacaaggcccccgaggccatcaattacgagcagatcaagaaggatctggccgaggagctgaccttcgatatcgactataagacatccgaggtgaaccagcgggtgttttctctggacgaggtgtttgagatcgccaatttcaacaattacctgaaccagtccggcatcaccaagttcaatacaatcatcggcggcaagtttgtgaacggcgagaataccaagagaaagggcatcaacgagtacatcaatctgtatagccagcagatcaacgacaagaccctgaagaagtacaagatgagcgtgctgttcaagcagatcctgtccgatacagagtctaagagctttgtgatcgataagctggaggatgactctgacgtggtgaccacaatgcagagcttttatgagcagatcgccgccttcaagaccgtggaggagaagtctatcaaggagacactgagcctgctgttcgatgacctgaaggcccagaagctggacctgtctaagatctacttcaagaacgataagtccctgaccgacctgtctcagcaggtgtttgatgactatagcgtgatcggcaccgccgtgctggagtacatcacacagcagatcgccccaaagaacctggataatccctctaagaaggagcaggagctgatcgccaagaagaccgagaaggccaagtatctgagcctggagacaatcaagctggccctggaggagttcaataagcaccgggatatcgacaagcagtgcagatttgaggagatcctggccaacttcgccgccatccccatgatctttgatgagatcgcccagaacaaggacaatctggcccagatctccatcaagtaccagaaccagggcaagaaggacctgctgcaggcctctgccgaggatgacgtgaaggccatcaaggatctgctggaccagaccaacaatctgctgcacaagctgaagatcttccacatctcccagtctgaggataaggccaatatcctggataaggacgagcacttttatctggtgttcgaggagtgttacttcgagctggccaacatcgtgcccctgtacaacaagatcagaaattatatcacacagaagccttactccgacgagaagtttaagctgaacttcgagaacagcaccctggccaacggctgggataagaataaggagcctgacaacacagccatcctgttcatcaaggatgacaagtactatctgggcgtgatgaataagaagaacaataagatcttcgatgacaaggccatcaaggagaacaagggcgagggctacaagaagatcgtgtataagctgctgcccggcgccaataagatgctgcctaaggtgttcttttccgccaagtctatcaagttctacaacccatccgaggacatcctgcggatcagaaatcactccacccacacaaagaacggctctccccagaagggctatgagaagtttgagttcaatatcgaggattgccggaagtttatcgacttctacaagcagagcatctccaagcaccctgagtggaaggattttggcttcaggtttagcgacacccagcggtacaactccatcgacgagttctacagagaggtggagaatcagggctataagctgacatttgagaacatctctgagagctacatcgacagcgtggtgaatcagggcaagctgtacctgttccagatctataacaaggacttcagcgcctattccaagggccggccaaacctgcacaccctgtactggaaggccctgttcgatgagagaaatctgcaggacgtggtgtataagctgaacggcgaggccgagctgttttacaggaagcagtccatccctaagaagatcacacacccagccaaggaggccatcgccaacaagaataaggacaatcctaagaaggagagcgtgttcgagtacgatctgatcaaggacaagcggttcaccgaggataagttctttttccactgtccaatcacaatcaacttcaagtcctctggcgccaacaagtttaatgacgagatcaatctgctgctgaaggagaaggccaacgatgtgcacatcctgagcatcgaccggggcgagagacacctggcctactataccctggtggatggcaagggcaatatcatcaagcaggataccttcaacatcatcggcaatgacaggatgaagacaaactaccacgataagctggccgccatcgagaaggatagggactccgcccgcaaggactggaagaagatcaacaatatcaaggagatgaaggagggctatctgtctcaggtggtgcacgagatcgccaagctggtcatcgagtacaatgccatcgtggtgttcgaggatctgaacttcggctttaagaggggccgctttaaggtggagaagcaggtgtatcagaagctggagaagatgctgatcgagaagctgaattacctggtgtttaaggataacgagttcgacaagaccggaggcgtgctgagggcataccagctgaccgccccctttgagacattcaagaagatgggcaagcagacaggcatcatctactatgtgccagccggccgcacctccaagatctgccccgtgacaggctttgtgaaccagctgtaccctaagtatgagtccgtgtctaagagccaggagtttttcagcaagttcgataagatctgttataatctggacaagggctacttcgagttttccttcgattataagaactttggcgacaaggccgccaagggcaagtggaccatcgcctctttcggcagccggctgatcaactttagaaattccgataagaaccacaattgggacacccgggaggtgtacccaacaaaggagctggagaagctgctgaaggactacagcatcgagtatggccacggcgagtgcatcaaggccgccatctgtggcgagagcgataagaagtttttcgccaagctgacctccgtgctgaatacaatcctgcagatgcggaacagcaagaccggcacagagctggactacctgatctcccccgtggccgatgtgaacggcaacttcttcgacagcagacaggcccccaagaatatgcctcaggatgccgacgccaacggcgcctatcacatcggcctgaagggcctgatgctgctgggcaggatcaagaacaatcaggagggcaagaagctgaacctggtcatcaagaacgaggagtactttgagttcgtgcagaaccgcaacaattga

(19) United States
(12) Patent Application Publication
(10) Pub. No.: US 2026/0028605 A1
(43) Pub. Date: Jan. 29, 2026

(54) GENE EDITING PROTEIN VARIANT CAPABLE OF REDUCING GENE EDITING OFF-TARGET RATE

(71) Applicant: SHANGHAI TOLO BIOTECHNOLOGY COMPANY LIMITED, Shanghai (CN)

(72) Inventors: Lei YIN, Shanghai (CN); Jin WANG, Shanghai (CN)

(21) Appl. No.: 18/865,224

(22) PCT Filed: Mar. 31, 2023
(86) PCT No.: PCT/CN2023/085720

§ 371 Date: Sep. 8, 2025

(30) Foreign Application Priority Data
May 10, 2022 (CN) 202210508434.7

(51) Int. Cl. C12N 9/22; A61K 31/7105; A61K 38/46; C12N 15/11; C12N 15/90

(52) U.S. Cl. CPC C12N 9/226; A61K 31/7105; A61K 38/465; C12N 15/11; C12N 15/902; C12N 2310/20

(57) ABSTRACT

A gene editing protein variant is capable of reducing a gene editing off-target rate. The variant is an unnatural protein with cis-cleavage activity, and the variant has reduced trans-cleavage activity as compared to a wild-type gene editing protein thereof. Furthermore, the variant is mutated at one or more of cleavage activity-related core amino acid sites of the wild-type gene editing protein selected from the following: a phenylalanine (F) site corresponding to the 1081st position of FnCas12a; and/or a lysine (K) site corresponding to the 1069th site of the FnCas12a. The variant can have cis-cleavage activity and reduced trans-cleavage activity. Moreover, the gene editing protein variant or a gene editing system having the gene editing protein variant can significantly reduce the gene editing off-target rate.

### INCORPORATION OF SEQUENCE LISTING

This application contains a sequence listing submitted in Computer Readable Form (CRF). The CRF file contains the sequence listing entitled “1-2-PBA4080226-SequenceListing.xml”, which was created on Sep. 8, 2025, and is 25,518 bytes in size. The information in the sequence listing is incorporated herein by reference in its entirety.

### TECHNICAL FIELD

The present invention relates to the field of biotechnology, specifically to a gene editing protein variant capable of reducing a gene editing off-target rate.

### BACKGROUND

Gene editing refers to the deletion, insertion, or substitution, etc., of DNA sequences, and is widely used in gene function research, disease model establishment, disease treatment, and transgenic animal and plant engineering, etc. The first generation of gene editing technology is based on Zinc Finger Nuclease (ZFN). ZFN contains a DNA-binding zinc finger domain that can specifically recognize sequences, and by modifying this domain, it is possible to target different DNA sequences. A DNA-binding zinc finger domain typically consists of multiple zinc finger structures, each recognizing 3 bases. Therefore, the target sequence of a ZNF must be a multiple of 3. Due to the context-dependent effect of the recognition domain in ZNFs, their design and screening are extremely challenging, limiting their application range. Furthermore, this technology has several drawbacks, such as high costs, labor-intensive processes, long duration, low success rates, susceptibility to off-target effects, and significant cytotoxicity. The second generation of gene editing technology is based on Transcription Activator-like Effector Nuclease (TALEN). The specific unit module in TALEN for recognizing the target site DNA is a pair of amino acids separated by 32 constant amino acid residues. Different pairs of amino acids can correspond one-to-one with the four nucleotide bases A, G, T, and C. The corresponding pair of amino acid sequences is deduced based on the sequence of the target DNA, forming the TALEN target recognition module. The assembly of this module requires a large number of molecular cloning and sequencing operations, which limits the widespread adoption of this technology.

The third generation of gene editing technology is based on CRISPR-Cas technology, which achieves specific recognition of target DNA sequences through guide RNAs. The workload involved in the design and synthesis of guide RNAs is significantly less than that required for constructing the DNA recognition modules in TALEN and ZFN technologies. Guide RNAs can bind to Cas proteins with nuclease activity and direct them to cleave the target DNA.

Currently, gene editing proteins still exhibit a certain degree of off-target rate. When the gene editing protein (such as Cas12a) forms a ternary complex with the guide RNA and target DNA, it not only exhibits cis-cleavage activity towards the target DNA but also demonstrates nonspecific trans-cleavage activity towards the single-stranded DNA present in the system. When DNAs are in a state of replication or transcription, the double-stranded DNAs will unwind into single-stranded DNAs. At this point, the trans-cleavage activity of gene editing proteins (such as Cas12a) may lead to the cleavage of these DNAs, resulting in off-target effects and causing cytotoxicity issues. Thus, it is necessary to eliminate the trans-cleavage activity of gene editing proteins (such as Cas12a) in order to address the cytotoxicity issues caused by off-target effects.

Therefore, there is an urgent need in this field to develop methods for eliminating the trans-cleavage activity of gene editing proteins (such as Cas12a) in order to address the cytotoxicity issues caused by off-target effects.

### SUMMARY OF THE INVENTION

The purpose of the present invention is to provide a method for eliminating the trans-cleavage activity of gene editing proteins (such as Cas12a) in order to address the cytotoxicity issues caused by off-target effects.

In the first aspect of the present invention, it provides a gene editing protein variant, which is an unnatural protein with cis-cleavage activity, and the variant has reduced trans-cleavage activity as compared to a wild-type gene editing protein thereof, and the variant undergoes mutation in the wild-type gene editing protein at one or more core amino acid sites selected from the group consisting of:
a phenylalanine (F) site corresponding to the 1081st position of FnCas12a; and/or
a lysine (K) site corresponding to the 1069th position of FnCas12a.

In another preferred embodiment, the expression “the variant has reduced trans-cleavage activity as compared to a wild-type gene editing protein thereof” refers to a reduction in the trans-cleavage activity of the variant by ≥50%, preferably ≥80%, more preferably ≥90% or 100%, when compared to the wild-type gene editing protein.

In another preferred embodiment, the phenylalanine (F) at the 1081st position of FnCas12a is mutated to one or more amino acids selected from the group consisting of: arginine (R), tyrosine (Y), tryptophan (W), glutamine (Q), asparagine (N), lysine (K), glutamic acid (E), aspartic acid (D), and a combination thereof.

In another preferred embodiment, the lysine (K) at the 1069th position of FnCas12a is mutated to one or more amino acids selected from the group consisting of: arginine (R), tyrosine (Y), glutamine (Q), asparagine (N), lysine (K), glutamic acid (E), aspartic acid (D), and a combination thereof.

In another preferred embodiment, the phenylalanine (F) at the 1081st position of FnCas12a is mutated to arginine (R).

In another preferred embodiment, the lysine (K) at the 1069th position of FnCas12a is mutated to arginine (R).

In another preferred embodiment, the mutation is selected from the group consisting of: F1081R, K1069R, and a combination thereof.

In another preferred embodiment, the gene editing protein is a type-V CRISPR/Cas protein.

In another preferred embodiment, the gene editing protein is selected from the group consisting of: Cas12, Cas14, and a combination thereof.

In another preferred embodiment, the gene editing protein is selected from the group consisting of: Cas12a, Cas12b, Cas12e, and a combination thereof.

In another preferred embodiment, the Cas12a is selected from the group consisting of: FnCas12a, LbCas12a, ErCas12a, EvCas12a, Lb5Cas12a, HkCas12a, OsCas12a, TsCas12a, BbCas12a, BoCas12a, Lb4Cas12a, CeCas12a, PrCas12a, CsbCas12a, BhCas12a, SsCas12a, Lb3Cas12a, BpCas12a, PdCas12a, BfCas12a, PcCas12a, cMtCas12a, PeCas12a, LiCas12a, Lb2Cas12a, PmCas12a, MbCas12a, EeCas12a, CsbCas12a, ArCas12a, BsCas12a, AbCas12a, AsCas12a, and a combination thereof.

In another preferred embodiment, the origin of the Cas12a is selected from the group consisting of: Leptotrichia, Listeria, Corynebacterium, Sutterella, Legionella, Treponema, Filifactor sp., Eubacterium, Streptococcus, Lactobacillus, Mycoplasma, Bacteroides sp., Flaviivola, Flavobacterium, Azospirillum, Sphaerochacta, Gluconacetobacter, Neisseria, Rothia, Parvibaculum, Staphylococcus, Nitratifractor, Mycoplasma, Campylobacter, Lachnospiraceac, and a combination thereof.

In another preferred embodiment, the origin of the Cas12a is selected from the group consisting of: Francisella tularensis (FnCas12a), Acidaminococcus sp. BV3L6 (AsCas12a), Lachnospiraceae bacterium ND2006 (LbCas12a), Lachnospiraceae bacterium NC2008 (Lb5Cas12a), Helcococcus sp kunzii (HkCas12a), Oribacterium sp. NK2B42 (OsCas12a), Thiomicrospira sp. XS5 (TsCas12a), Bacteroidales bacterium KA00251 (BbCas12a), Bacteroidetes oral taxon 274 (BoCas12a), Lachnospiraceae bacterium MC2017 (Lb4Cas12a), Coprococcus eutactus (CeCas12a), Prevotella ruminicola strain BPI-34 (PrCas12a), candidatus Saccharibacteria bacterium (CsbCas12a), Butyrivibrio hungatei strain MB2003 (BhCas12a), Smithella sp. SC_K08D17 (SsCas12a), Lachnospiraceae bacterium MC2017 (Lb3Cas12a), Bytyrivibrio proteoclasticus (BpCas12a), Prevotella disens (PdCas12a), Butyrivibrio fibrisolvens MD2001 (BfCas12a), Porphyromonas crevioricanis (PcCas12a), candidatus Methanoplasma termitum (CMtCas12a), Peregrinibacteria bacterium (PeCas12a), Leptospira inadaiserovar Lyme (LiCas12a), Lachnospiraceae bacterium MA2020 (Lb2Cas12a), Porphyromonas macaca (PmCas12a), Moraxella bovoculi 237 (MbCas12a), Eubacterium eligens (EcCas12a), candidatus Saccharibacteria bacterium (CsbCas12a), Eubacte riumrectale (ErCas12a), Agathobacter rectalisstrain (ArCas12a), Butyrivibrio sp. NC3005 (BsCas12a), Arcobacter butzleri (AbCas12a), and a combination thereof.

In another preferred embodiment, the origin of the Cas12b is selected from the group consisting of: Alicyclobacillus kakegawensis, V3-13 species of Bacillus sp., Bacillus hisashii, Lentisphacria bacterium, Laceyella sediminis, and a combination thereof.

In another preferred embodiment, the Cas12b is selected from the group consisting of: AacCas12b, AaCas12b, BthCas12b, AapCas12b, AkCas12b, AmCas12b, Bs3Cas12b, LsCas12b, and a combination thereof.

In another preferred embodiment, the origin of the gene editing protein is selected from the group consisting of: Leptotrichia, Listeria, Corynebacterium, Sutterella, Legionella, Treponema, Filifactor sp., Eubacterium, Streptococcus, Lactobacillus, Mycoplasma, Bacteroides sp., Flaviivola, Flavobacterium, Azospirillum, Sphaerochaeta, Gluconacetobacter, Neisseria, Rothia, Parvibaculum, Staphylococcus, Nitratifractor, Mycoplasma, Campylobacter, Lachnospiraceae, and a combination thereof.

In another preferred embodiment, the origin of the gene editing protein is selected from the group consisting of: Lachnospiraceae bacterium ND2006 (LbCas12a), Thiomicrospira sp. XS5 (TsCas12a), Francisella tularensis (FnCas12a), Bacteroidetes oral taxon 274 (BoCas12a), Oribacterium sp. NK2B42 (OsCas12a), Acidaminococcus sp. BV3L6 (AsCas12a), Helcococcus sp kunzii (HkCas12a), Lachnospiraceae bacterium NC2008 (Lb5Cas12a), and a combination thereof.

In another preferred embodiment, the 1081st position and the 1069th position of FnCas12a are located at positions 1081 and 1069 of FnCas12a.

In another preferred embodiment, the 1081st position and the 1069th position of FnCas12a are located at positions 1019 and 1007 of BbCas12a.

In another preferred embodiment, the 1081st position and the 1069th position of FnCas12a are located at positions 1069 and 1057 of AsCas12a.

In another preferred embodiment, the 1081st position and the 1069th position of FnCas12a are located at positions 1033 and 1021 of BoCas12a.

In another preferred embodiment, the 1081st position and the 1069th position of FnCas12a are located at positions 1090 and 1078 of HkCas12a.

In another preferred embodiment, the 1081st position and the 1069th position of FnCas12a are located at positions 1004 and 992 of Lb4Cas12a.

In another preferred embodiment, the 1081st position and the 1069th position of FnCas12a are located at positions 980 and 968 of Lb5Cas12a.

In another preferred embodiment, the 1081st position and the 1069th position of FnCas12a are located at positions 1018 and 1006 of LbCas12a.

In another preferred embodiment, the 1081st position and the 1069th position of FnCas12a are located at positions 1001 and 989 of OsCas12a.

In another preferred embodiment, the 1081st position and the 1069th position of FnCas12a are located at positions 1070 and 1058 of TsCas12a.

In another preferred embodiment, the gene editing protein is FnCas12a.

In another preferred embodiment, the sequence of the gene editing protein is as shown in SEQ ID NO. 1.

In another preferred embodiment, the amino acid sequence of the variant is as shown in any one of SEQ ID NOs. 2-3.

In another preferred embodiment, the variant is a polypeptide having the amino acid sequence shown in any one of SEQ ID NOs.: 2-3, or an active fragment thereof, or a conserved variant polypeptide thereof.

In another preferred embodiment, the variant has an amino acid sequence that is identical or substantially identical to that of the wild-type gene editing protein, except for the mutation (such as at positions 1081 and/or 1069).

In another preferred embodiment, the “substantially identical” refers to having at most 50 (preferably 1-20, more preferably 1-10, more preferably 1-5) amino acid differences, wherein the differences comprise amino acid substitutions, deletions, or additions, and the variant has cis-cleavage activity and reduced trans-cleavage activity.

In another preferred embodiment, the homology of the variant to the wild-type gene editing protein is at least 80%, preferably at least 85% or 90%, more preferably at least 95%, and most preferably at least 98% or 99%.

In another preferred embodiment, the variant is selected from the group consisting of:
(a) a polypeptide having the amino acid sequence shown in any one of SEQ ID NOs.: 2-3;
(b) a polypeptide derived from (a) that is formed by the substitution, deletion, or addition of one or more (such as 2, 3, 4, or 5) amino acid residues within the amino acid sequence shown in any one of SEQ ID NOs.: 2-3, and has cis-cleavage activity and reduced trans-cleavage activity.

In another preferred embodiment, the homology of the derived polypeptide to the sequence shown in any one of SEQ ID NOs.: 2-3 is at least 60%, preferably at least 70%, more preferably at least 80%, and most preferably at least 90%, such as 95%, 97% or 99%.

In another preferred embodiment, the variant is formed by mutating the wild-type gene editing protein.

In the second aspect of the present invention, it provides a polynucleotide encoding the variant of the first aspect of the present invention.

In another preferred embodiment, the polynucleotide is selected from the group consisting of:
(a) a polynucleotide encoding a polypeptide as shown in any one of SEQ ID NOs. 2-3;
(b) a polynucleotide with a sequence as shown in any one of SEQ ID NOs.: 4-5;
(c) a polynucleotide with a nucleotide sequence that has ≥80% (preferably ≥90%, more preferably ≥95%, most preferably ≥98%) homology to the sequence shown in any one of SEQ ID NOs.: 4-5, and encodes a polypeptide as shown in any one of SEQ ID NOs.: 2-3;
(d) a polynucleotide complementary to any one of the polynucleotides described in (a)-(c).

In another preferred embodiment, the polynucleotide additionally comprises an auxiliary element flanking the ORF of the variant, wherein the auxiliary element is selected from the group consisting of: a signal peptide, a secretory peptide, a tag sequence (such as 6His), and a combination thereof.

In another preferred embodiment, the polynucleotide is selected from the group consisting of: a genomic sequence, a cDNA sequence, an RNA sequence, and a combination thereof.

In another preferred embodiment, the polynucleotide further comprises a promoter operationally linked to the ORF sequence of the variant.

In another preferred embodiment, the promoter is selected from the group consisting of: a constitutive promoter, a tissue-specific promoter, an inducible promoter, and a strong promoter.

In the third aspect of the present invention, it provides a vector comprising the polynucleotide of the second aspect of the present invention.

In another preferred embodiment, the vector comprises one or more promoters operationally linked to the nucleic acid sequence, enhancer, transcription termination signal, polyadenylation sequence, origin of replication, selectable marker, nucleic acid restriction site, and/or homologous recombination site.

In another preferred embodiment, the vector comprises a plasmid vector, a phage vector, a cosmid cloning vector, a phagemid vector, an artificial chromosome vector, an episomal vector, a viral vector, and a combination thereof.

In another preferred embodiment, the artificial chromosome vector is selected from the group consisting of: bacterial artificial chromosome (BAC), yeast artificial chromosome (YAC), P1 artificial chromosome (PAC), and a combination thereof.

In another preferred embodiment, the viral vector is selected from the group consisting of: a retroviral vector, an adenoviral vector, an adeno-associated viral vector, a herpesviral vector, a poxviral vector, a baculoviral vector, a papovavirus, a papillomavirus vector, an HBP Epstein Barrvirus vector, a vaccinia viral vector, a Semliki Forest virus (SFV), and a combination thereof.

Preferably, the vector is selected from the group consisting of: pcDNA, pTT, pTT3, pEFBOS, pBV, pJV, pBJ, pGEX, VSV, pBR322, pCMV-HA, pEN, YAC, BAC, λ phage, M13 phage, phagemid, pCAS9, pCEN6, pYES1L, p3HPRT1, pFN2A, pBC, pTZ, pGEM, pGEMK, pEX, pSAR, pCEP, comsid, pBluescript, pKJK, pFloxin, pCP, pHR, pUC, pMAL, pALTER, pBAD, pCa1, pL, pET, pGEMEX, pCI, pCMV, pEGFP, pEGFT, pSV2, pFUSE, pVITRO, pVIVO, pMONO, pSELECT, pUNO, pDUO, Psg5L, pBABE, pWPXL, pBI, p15TV-L, pPro18, pTD, pRS420, pLexA, pACT2.2, pRS403, pRS404, pRS405, pRS406, pRS413, pRS414, pRS415, pRS416, and a combination thereof.

In another preferred embodiment, the vector comprises a cloning vector, a transformation vector, an expression vector, a shuttle vector, an integration vector, and a multifunctional vector.

In the fourth aspect of the present invention, it provides a host cell comprising the vector of the third aspect of the present invention, or having the polynucleotide of the second aspect of the present invention integrated into its genome.

In another preferred embodiment, the host cell is a prokaryotic recipient cell.

In another preferred embodiment, further, the prokaryotic recipient cell is selected from the group consisting of: Escherichia coli, Lactic acid bacteria, Bacillus subtilis, Cyanobacteria, Streptomyces, Pseudomonas, Propionibacterium, Pectinatus sp., Bacteroides sp., Bacillus subtilis, Streptomyces sp., Anabaena, Arthrobacter, Agrobacterium, Acetobacter, Acetobacterium, Bacillus, Brevibacillus, Bifidobacterium, Brachybacterium, Brevibacterium, Carnobacterium, Xenorhabdus, Photorhabdus, Corynebacterium, Enterobacter, Pasteurella, Lactobacillus, Alcaligenes, Flavobacterium spp., Clostridium, Pasteuria, Escherichia, Gluconacetobacter, Gluconobacter, Hafnia, Halomonas, Klebsiella, Kocuria, Leucononstoc, Macrococcus, Methylomonas, Methylobacter, Methylocella, Methylococcus, Microbacterium, Micrococcus, Microcystis, Moorella, Oenococcus, Pediococcus, Prochlorococcus, Propionibacterium, Proteus, Pseudoalteromonas, Pseudomonas, Psychrobacter, Rhodobacter, Rhodococcus, Rhodopseudomonas, Erwinia, shigella, Serratia, Salmonella, Staphylococcus, Streptococcus, Streptomyces, Rhizobium, Synechococcus, Syncchocystis, Tetragenococcus, Weissella, Xanthomonas, Zymomonas, Rhodopseudomonas, and Salmonella typhimuium, Methylophilius, Azotobacter, Ensifer, Sphingomonas sp., Burkholderia, Candidatus Glomeribacter, Dyella, Herbaspirillum, Bradyrhizobium, Variovorax, Sphingobacterium, Zymomonas, Serratia, Acromonas, Vibrio, Desulfovibrio, Spirillum, Acetobacter, and a combination thereof.

In another preferred embodiment, further, the prokaryotic recipient cell is selected from the group consisting of: Escherichia coli, Rodhobacter sphacroides, Pseudoalteromonas haloplanktis, Shewanella sp., Strain Ac10, Pseudomonas fluorescens, Pseudomonas putida, Pseudomonas aeruginosa, P. alcaligenes, Pseudomonas aeruginosa PAO1-LAC, Pseudomonas putida KT2440, Halomonas elongata, Pseudoalteromonas citrea, Chromohalobacter salex'igens, Streptomyces lividans, Streptomyces griseus, Streptomyces coelicolor, S. avermitilis, S. griseus, S. scabies, S. lividans TK24, S. lividans 1326, Nocardia lactamdurans, Mycobacterium smegmatis, Corynebacterium glutamicum, Corynebacterium ammoniagenes, Brevibacterium lactofermentum, Arthrobacter nicotianae, Acetobacter aceti, Arthrobacter arilaitensis, Bacillus cereus, Bacillus coagulans, Bacillus sphacricus, Bacillus stearothermophilus, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, Lactobacillus gasseri, Lactobacillus acidifarinac, Lactobacillus yamanashiensis, Lactobacillus jensenii, Lactobacillus sakei, Gluconobacter oxydans, Bifidobacterium adolescentis, Brachybacterium tyrofermentans, Brevibacterium linens, Carnobacterium divergens, Corynebacterium flavescens, Gluconacetobacter europaeus, Gluconacetobacter johannae, Gluconobacter oxydans, Klebsiella oxytoca, Actinobacillus succinogenes, Mannhei succiniciproducers MBEL 55E, Bacteroides amylophilus, Microbacterium foliorum, Propionibacterium acidipropionici, Proteus vulgaris, Psychrobacter celer, Bifidobacterium bifidum/breve/longum, Hafnia alvei, Anaerobiospirillum succiniciproducens, Ruminococcus flavefaciens, Prevotella ruminicola, Succcinimonas amylolytica, Succinivibrio dextrinisolvens, Wolinella succinogenes, Cytophaga succinicans, Rhizobium etli, Zymomonas mobilis, Clostridium acetobutylicum, Clostridium ljungdahlii/aceticum/acetobutylicum/beijerinckii/butyricum, Enterococcus faccium, Micrococcus lylae, Oenococcus oeni, Macrococcus cascolyticus, Pediococcus acidilactici, Staphylococcus condimenti, Streptococcus thermophilus, Tetragenococcus halophilus, Kocuria rhizophila, Leuconostoc citreum, Weissella cibaria, Weissella koreensis, Moorella themocellum/thermoacetica, Bacillus thuringiensis, and a combination thereof.

In another preferred embodiment, further, the Escherichia coli is selected from the group consisting of: BL21, BL21 (DE3), W3110, MG1655, RB791, RV308, HMS 174, HMS174 (DE3), NM533, XL1-Bluc, C600, DH1, HB101, JM109, Top10, DH5α, DH10β, TG1, BW23473, BW23474, MW003, MW005 cells, and a combination thereof; wherein the Bacillus megaterium is selected from the group consisting of: QMB1551, PV361, DSM319, and a combination thereof.

In another preferred embodiment, the host cell is a eukaryotic cell.

In another preferred embodiment, further, the eukaryotic recipient cell is selected from the group consisting of: a yeast, a fungus, a plant cell, an animal cell, and a combination thereof.

In another preferred embodiment, further, the yeast is selected from the following species: Rhodotorula spp., Aurcobasidium spp., Saccharomyces spp., Sporobolomyces spp., and a combination thereof.

In another preferred embodiment, further, the yeast is selected from the group consisting of: Saccharomyces cerevisiae, Schizosaccharomyces pombe, Kluyveromyces lactis, Kluyveromyces fragilis (ATCC 12,424), Kluyveromyces marxianus var. bulgaricus (ATCC 16,045), Kluyveromyces wickerhamii (ATCC 24,178), Kluyveromyces waltii (ATCC 56,500), Kluyveromyces waltii (K. waltii) (ATCC 56500), Kluyveromyces drosophilarum (ATCC 36,906), Kluyveromyces thermotolerans, Kluyveromyces marxianus, Pichia pastoris, P. methanolica, P. Stipitis, Yarrowia lipolytica, Candida sp., Schwanniomyces occidentalis, Hansenula polymorpha, Saccharomyces carlsbergensis, Saccharomyces diastaticus, Debaryomyces hansenii, Saccharomyces kluyveri, Candida norvegica, Saccharomyces oviformis, and a combination thereof.

In another preferred embodiment, further, the fungus is a filamentous fungus, wherein the filamentous fungus is selected from the group consisting of: Acremonium, Aspergillus, Aureobasidium, Bjerkandera, Ceriporiopsis, Chrysosporium, Coprinus, Coriolus, Cryptococcus, Filibasidium, Fusarium, Humicola, Piricularia, Magnaporthe, Mucor, Myceliophthora, Neocallimastix, Neurospora, Paccilomyces, Penicillium, Phanerochaete, Phlebia, Piromyces, Pleurotus, Schizophyllum, Talaromyces, Talaromyces, Thermoascus, Thiclavia, Tolypocladium, Trametes, Trichoderma, Fusarium, Humicola, Neurspora, Scytalidium, Hypocrea, Chrysosporium, Filibasidium, Gibberella, Magnaporthe, Mucor, Myceliophthora, Myrothecium, Neocallimastix, and a combination thereof.

In another preferred embodiment, further, the fungus is selected from the group consisting of: Aspergillus terreus, A. oryzae, Aspergillus niger, A. awamori, Aspergillus nidulans, Aspergillus fumigatus, Aspergillus aculeatus, Aspergillus clavatus, Aspergillus flavus, Aspergillus foctidus, Aspergillus japonicus, A. oryzae, Rhizopus arrhizus, Rhizopus oryzae, Trichoderma reesei, Trichoderma reesei QM9414, Trichoderma reesei RUT-C30, Trichoderma reesei QM6a, T. atroviride, T. harzianum, T. virens, T. asperellum, T. longibrachiatum, Trichoderma koningii, Trichoderma longibrachiatum, Trichoderma atroviride, Trichoderma virens, Trichoderma viride, Paccilomyces varioti, Penicillium viniferum, P. purpurogenum, P. funiculosum, Penicillium (Talaromyces) emersonii, P. camemberti, P. roqueforti, Phanerochacte chrysosporium, Ashbya gossypii, Byssochlamys nivea, Bjerkandera adusta, Ceriporiopsis aneirina, Ceriporiopsis caregica, Ceriporiopsis gilvescens, Ceriporiopsis pannocinta, Ceriporiopsis rivulosa, Ceriporiopsis subrufa, Ceriporiopsis subvermispora, Chrysosporium lucknowense, Chrysosporium inops, Chrysosporium keratinophilum, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roscum, Fusarium sambucinum, Fusarium sarcochroum, Fusarium sporotrichioides, Fusarium sulphureum, Fusarium torulosum, Fusarium trichothecioides, Fusarium venetum, Humicola insolens, Humicola lanuginosa, Mucor miehei, Myceliophthora thermophila, Neurospora crassa, Penicillium purpurogenum, Phanerochacte chrysosporium, Phlebia radiata, Pleurotus eryngii, Thiclavia terrestris, Trametes villosa, Trametes versicolor, and a combination thereof.

In another preferred embodiment, further, the plant cell is a dicotyledonous plant cell or a monocotyledonous plant cell. Further, preferably, the dicotyledonous plant cell is selected from the group consisting of: Glycine max (soybean) cell, Helianthus annuus (sunflower) cell, Solanum lycopersicum (tomato) cell, Brassica genus cell, Gossypium (cotton) cell, Beta vulgaris (sugar beet) cell, Nicotiana (tobacco) cell, Solanum tuberosum (potato) cell, Petunia (morning glory) cell, Arabidopsis thaliana cell, and a combination thereof. The monocotyledonous plant cell is selected from the group consisting of: Hordeum vulgare (barley) cell, Maize cell, Corn cell, Avena sativa (oat) cell, Oryza sativa (rice) cell, Sorghum bicolor (Sorghum) cell, Saccharum officinarum (sugarcane) cell, Triticum aestivum (wheat) cell, and a combination thereof.

In another preferred embodiment, further, the animal cell is an insect cell or a mammalian cell.

In another preferred embodiment, further, the insect cell is selected from the group consisting of: Autographa californica (alfalfa looper), Spodoptera frugiperda (fall army worm), Spodoptera exigua (beet armyworm), Trichoplusia ni (cabbagelooper), Lymantria dispar (gypsy moth), Bombyx mori (silkworm), Anticarsia gemmatalis (velvetbeancaterpillar), Heliothis virescens (tobacco budworm), Heliothis subflexa (Subflexus straw moth), Mamestra brassicae (cabbage moth), Helicoverpa armigera (cotton bollworm), Helicoverpa zea (corn earworm), Agrotis ipsilon (black cutworm), Anagrapha falcifera (celery looper), Galleria mellonella (honeycomb moth), Rachiplusia ou (graylooper), Plutella xylostella (diamondback moth), Drosophila melanogaster (drosophila), Aedes aegypti (mosquito), and a combination thereof.

In another preferred embodiment, further, the insect cell is selected from the group consisting of: Sf9 cell from Spodoptera frugiperda, Sf21 cell from Spodoptera frugiperda, High-Five cell from Trichoplusia ni (identical to Hi5 and High-Five BTI-TN-5B1-4), Tn-368 cell from Trichoplusia ni, Se301 cell from Spodoptera exigua, S2 cell from Drosophila melanogaster, Bm5 cell from Bombyx mori, Ld652Y from Lymantria dispar, LdEIta from Lymantria dispar, and a combination thereof.

In another preferred embodiment, further, the mammalian cell is selected from the group consisting of: SV40-transformed monkey kidney cell line CV1 (COS-7, ATCC CRL 1651); human embryonic kidney cell line (HEK293 or suspension culture-derived 293 cell subclone, Graham et al., J. Gen Virol. 36:59 (1977)), such as Expi293; baby hamster kidney cell (BHK, ATCC CCL 10); baby hamster kidney cell (BHK); Chinese hamster ovary cell/-DHFR (CHO, Urlaub et al., Proc. Natl. Acad. Sci. USA 77:4216 (1980)); mouse testis sertoli cell (TM4, Mather, Biol. Reprod. 23:243-251 (1980)); monkey kidney cell (CV1 ATCC CCL 70); African green monkey kidney cell (VERO-76, ATCC CRL-1587); human cervical carcinoma cell (HELA, ATCC CCL 2); canine kidney cell (MDCK, ATCC CCL 34); Buffalo rat liver cell (BRL 3A, ATCC CRL 1442); human lung cell (W138, ATCC CCL 75); human liver cell (HepG2, HB 8065); mouse mammary tumor (MMT 060562, ATCC CCL51); TRI cell (Mather et al., Annals N.Y. Acad. Sci. 383:44-68 (1982)); MRC 5 cell; FS4 cell; CHO cell; NSO cell; myeloma cell lines such as YB 2/0, YO, NS0, P3X63, and Sp2/0, etc.; lymphocytes (e.g., Y0, NS0, Sp20 cells); and a combination thereof.

In another preferred embodiment, further, the mammalian cell is selected from a human cell, wherein the human cell is selected from the group consisting of: HeLa, Huh7, HEK293, HepG2, KATO-III, IMR32, MT-2, pancreatic β-cell, keratinocyte, bone marrow fibroblast, CHP212, primary neural cell, W12, SK-N-MC, Saos-2, WI38, primary hepatocyte, FLC4, 143TK-, DLD-1, embryonic lung fibroblast, primary foreskin fibroblast, Saos-2 osteosarcoma, MRC5, MG63 cell, and a combination thereof.

In the fifth aspect of the present invention, it provides a method for preparing a gene editing protein variant, comprising the steps of:
(a) culturing the host cell of the fourth aspect of the present invention under suitable conditions for expression, thereby expressing the gene editing protein variant; and
(b) isolating the gene editing protein variant.

In the sixth aspect of the present invention, it provides an enzyme formulation comprising the gene editing protein variant of the first aspect of the present invention.

In another preferred embodiment, the enzyme formulation comprises an injection and/or a freeze-dried formulation.

In the seventh aspect of the present invention, it provides a gene editing system, comprising:
the gene editing protein variant of the first aspect of the present invention, or a coding gene thereof, or an expression vector thereof; and
optionally, a guide RNA or an expression vector thereof, and/or an oligonucleotide or nucleic acid fragment or plasmid thereof used for target site break repair.

In another preferred embodiment, the expression vector comprises a plasmid and a viral vector.

In another preferred embodiment, the guide RNA comprises crRNA, tracrRNA, and sgRNA.

In another preferred embodiment, the guide RNA comprises unmodified and modified gRNAs.

In another preferred embodiment, the modified guide RNA comprises a chemical modification of bases.

In another preferred embodiment, the chemical modification comprises methylation modification, methoxy modification, fluorination modification, or thio modification.

In another preferred embodiment, the gene editing comprises CRISPR-based gene editing.

In the eighth aspect of the present invention, it provides a gene editing reagent comprising the gene editing protein variant of the first aspect of the present invention.

In another preferred embodiment, the reagent further comprises the following reagents: a guide RNA, or a vector for generating the guide RNA, and/or an oligonucleotide or nucleic acid fragment or plasmid thereof used for target site break repair.

In the ninth aspect of the present invention, it provides a composition, comprising:
the gene editing protein variant of the first aspect of the present invention, or the system of the seventh aspect of the present invention, or the gene editing reagent of the eighth aspect of the present invention; and
a pharmaceutically acceptable carrier.

In another preferred embodiment, the composition comprises a pharmaceutical composition.

In another preferred embodiment, the dosage form of the composition is selected from the group consisting of: a freeze-dried formulation, a liquid formulation, and a combination thereof.

In another preferred embodiment, the dosage form of the composition is a liquid formulation.

In another preferred embodiment, the dosage form of the composition is an injection.

In another preferred embodiment, the composition is a cell formulation.

In another preferred embodiment, the expression vector of the gene editing protein variant and the expression vector of the guide RNA are the same vector or different vectors.

In another preferred embodiment, in the composition, the system of the third aspect of the present invention accounts for 1-99 wt %, preferably 10-90 wt %, and more preferably 30-70 wt % of the total weight of the composition.

In the tenth aspect of the present invention, it provides a product combination, comprising:
the gene editing protein variant of the first aspect of the present invention, or the system of the seventh aspect of the present invention, or the gene editing reagent of the eighth aspect of the present invention.

In another preferred embodiment, the product combination further comprises: a guide RNA, or a vector for generating the guide RNA, and/or an oligonucleotide or nucleic acid fragment or plasmid thereof used for target site break repair.

In another preferred embodiment, the product combination further comprises a pharmaceutically acceptable carrier.

In the eleventh aspect of the present invention, it provides a kit, comprising: the gene editing protein variant of the first aspect of the present invention, or the enzyme formulation of the sixth aspect of the present invention, or the gene editing system of the seventh aspect of the present invention, or the gene editing reagent of the eighth aspect of the present invention, or the composition of the ninth aspect of the present invention, or the product combination of the tenth aspect of the present invention.

In another preferred embodiment, the kit further comprises a label or instructions.

In the twelfth aspect of the present invention, it provides a medical kit, comprising:
a first container, and the gene editing protein variant of the first aspect of the present invention, or the enzyme formulation of the sixth aspect of the present invention, or the gene editing system of the seventh aspect of the present invention, or the gene editing reagent of the eighth aspect of the present invention, or the composition of the ninth aspect of the present invention, or the product combination of the tenth aspect of the present invention, or a drug comprising the gene editing protein variant of the first aspect of the present invention, or the enzyme formulation of the sixth aspect of the present invention, or the gene editing system of the seventh aspect of the present invention, or the gene editing reagent of the eighth aspect of the present invention, or the composition of the ninth aspect of the present invention, or the product combination of the tenth aspect of the present invention, located in the first container.

In another preferred embodiment, the drug in the first container is a single-ingredient formulation comprising the gene editing protein variant of the first aspect of the present invention, or the enzyme formulation of the sixth aspect of the present invention, or the gene editing system of the seventh aspect of the present invention, or the gene editing reagent of the eighth aspect of the present invention, or the composition of the ninth aspect of the present invention, or the product combination of the tenth aspect of the present invention.

In another preferred embodiment, the dosage form of the drug is selected from the group consisting of: a freeze-dried formulation, a liquid formulation, and a combination thereof.

In another preferred embodiment, the dosage form of the drug is an oral form or injectable form.

In another preferred embodiment, the medical kit further comprises instructions.

In the thirteenth aspect of the present invention, it provides a medical kit, comprising:
(a1) a first container, and the gene editing protein variant of the first aspect of the present invention, or a coding gene thereof, or an expression vector thereof, or a drug comprising the gene editing protein variant of the first aspect of the present invention, or a coding gene thereof, or an expression vector thereof, located in the first container;
(b1) optionally, a second container, and a guide RNA or an expression vector thereof, or a drug comprising a guide RNA or an expression vector thereof, located in the second container.

In another preferred embodiment, the first container and the second container are different containers.

In another preferred embodiment, the drug in the first container is a single-ingredient formulation comprising the gene editing protein variant of the first aspect of the present invention, or a coding gene thereof, or an expression vector thereof.

In another preferred embodiment, the drug in the second container is a single-ingredient formulation comprising the guide RNA or an expression vector thereof.

In another preferred embodiment, the dosage form of the drug is selected from the group consisting of: a freeze-dried formulation, a liquid formulation, and a combination thereof.

In another preferred embodiment, the dosage form of the drug is an oral form or injectable form.

In another preferred embodiment, the medical kit further comprises instructions.

In the fourteenth aspect of the present invention, it provides a use of the gene editing protein variant of the first aspect of the present invention, or the enzyme formulation of the sixth aspect of the present invention, or the gene editing system of the seventh aspect of the present invention, or the gene editing reagent of the eighth aspect of the present invention, or the composition of the ninth aspect of the present invention, or the product combination of the tenth aspect of the present invention, or the medical kit of the thirteenth or fourteenth aspect of the present invention, in the manufacture of a reagent or kit for reducing a gene editing off-target rate.

In another preferred embodiment, the reagent or kit is used for reducing the trans-cleavage activity of the gene editing.

In another preferred embodiment, the reagent or kit is used for reducing the trans-cleavage activity of the gene editing while retaining the cis-cleavage activity.

In another preferred embodiment, the expression “reducing the trans-cleavage activity of the gene editing” refers to reducing the trans-cleavage activity of the gene editing by ≥80%, preferably ≥90% or 100%.

In the fifteenth aspect of the present invention, it provides a method for reducing a gene editing off-target rate, comprising the step of:
in the presence of the gene editing protein variant of the first aspect of the present invention, or the enzyme formulation of the sixth aspect of the present invention, or the gene editing system of the seventh aspect of the present invention, or the gene editing reagent of the eighth aspect of the present invention, or the composition of the ninth aspect of the present invention, or the product combination of the tenth aspect of the present invention, or the medical kit of the thirteenth or fourteenth aspect of the present invention, performing gene editing on a cell, thereby reducing the gene editing off-target rate.

In another preferred embodiment, the cell is a prokaryotic cell or a eukaryotic cell.

In another preferred embodiment, the cell is a mammalian cell.

In another preferred embodiment, the mammalian cell is a cell from a non-human mammal, such as a primate, cow, sheep, pig, dog, rodent, or Leporidae, for example, a cell from a monkey, heifer, sheep, pig, dog, rabbit, rat, or mouse.

In another preferred embodiment, the cell is a non-mammalian eukaryotic cell, such as a cell from a poultry bird (e.g., chicken), vertebrate fish (e.g., salmon), or crustacean (e.g., oyster, clam, lobster, shrimp).

In another preferred embodiment, the cell is a plant cell.

In another preferred embodiment, the plant cell is a cell from a monocotyledonous plant or dicotyledonous plant, or a cell from a cultivated plant or food crop, such as cassava, corn, Sorghum, soybean, wheat, oat, or rice.

In another preferred embodiment, the plant cell is a cell from algae, trees, or productive plants, fruits, or vegetables (such as tree species like citrus trees, e.g., orange trees, grapefruit trees, or lemon trees; peach trees or nectarine trees; apple trees or pear trees; nut trees, e.g., apricot trees, walnut trees, or pistachio trees; solanaceous plants; brassica plants; lactuca plants; spinacia plants; capsicum plants; cotton, tobacco, asparagus, carrots, kale, broccoli, cauliflower, tomatoes, eggplants, peppers, lettuce, spinach, strawberries, blueberries, raspberries, blackberries, grapes, coffee, cacao, and so on).

In another preferred embodiment, the gene editing is performed in an in vitro reaction system.

In another preferred embodiment, the method is non-diagnostic and non-therapeutic.

In another preferred embodiment, the cell is an in vitro cell.

It should be understood that within the scope of the present invention, the above-mentioned technical features of the present invention and the technical features specifically described in the following (such as the examples) can be combined with each other to form a new or preferred technical solution, which is not redundantly repeated one by one herein due to space limitation.

### DESCRIPTION OF THE DRAWINGS

FIG. 1 is a gel purification image of gene editing proteins, demonstrating the molecular weights of three gene editing proteins, all of which are 150 KDa. Lanes 1 and 2 are wild-type FnCas12a, with sample loading amounts of 3 μg and 5 μg, respectively; lanes 3 and 4 are mutant protein FnCas12a^{K1069R}, with sample loading amounts of 2 μg and 3 μg, respectively; lanes 5 and 6 are mutant protein FnCas12a^{F1081R}, with sample loading amounts of 2 μg and 3 μg, respectively.

FIG. 2 is an electrophoresis image of the products of the cis-cleavage reaction of gene editing proteins with target dsDNA, demonstrating that all three proteins possess cis-cleavage activity, and there is no significant difference in the cis-cleavage activity between the mutant proteins FnCas12a^{K1069R} and FnCas12a^{F1081R} and the wild-type gene editing protein. In FIG. 2, M represents a 1 Kb DNA marker; S represents the target dsDNA fragment, with a size of 829 bp; P represents the cis-cleavage products of the target dsDNA, with sizes of 529 bp and 300 bp, respectively.

FIG. 3 is a plot of the fluorescence signal changes of the trans-cleavage reaction of gene editing proteins with non-target ssDNA. As shown in FIG. 3, the fluorescence signal of the reaction system was detected using a real-time fluorescence quantitative PCR instrument, wherein the control represents a negative control trans-cleavage reaction system, which means that no target dsDNA is added to the trans-cleavage system. As time increased, no fluorescence signal was detected in the control system. WT represents the wild-type FnCas12a protein, and the fluorescence signal of its trans-cleavage reaction system increased with the extension of the reaction time, indicating that the wild-type FnCas12a has trans-cleavage activity. The fluorescence signals of the trans-cleavage reaction systems of the mutant proteins FnCas12a^{K1069R} and FnCas12a^{F1081R} remained at the background level and did not change with the extension of the reaction time, indicating that the mutant proteins FnCas12a^{K1069R} and FnCas12a^{F1081R} do not have significant trans-cleavage activity.

FIG. 4a-4e is an amino acid sequence alignment analysis chart for 10 types of Cas12a proteins. From this chart, it can be deduced that these 10 types of Cas12a protein amino acid sequences exhibit a high degree of homology.

FIG. 5 is a phylogenetic tree of CRISPR Type V Cas proteins (i.e., Cas12 proteins). According to the figure, all Cas12 proteins contain a RuvC functional domain. (Yan Winston X, et al. Functionally diverse type V CRISPR-Cas systems. [J]. Science (New York, N.Y.), 2018, 363 (6422).).

FIG. 6 is a schematic diagram of the protein domains of FnCas12a, indicating the start and end positions of the amino acid residues for each functional domain (Stefano, Stella, Pablo, et al. Conformational Activation Promotes CRISPR-Cas12a Catalysis and Resetting of the Endonuclease Activity. [J]. Cell, 2018, 175:1856-1871).

FIG. 7 is a schematic diagram of the protein domains of Cas12a, Cas12b, and Cas12e (Tong Baisong et al. The Versatile Type V CRISPR Effectors and Their Application Prospects [J]. Frontiers in Cell and Developmental Biology, 2021, 8:622103-622103.).

### DETAILED DESCRIPTION

After extensive and in-depth research, the inventors originally attempted to mutate the effector proteins of the Type V family in order to enhance their interaction with the trans-cleavage active substrate DNA. However, after extensive screening, a gene editing protein variant was unexpectedly obtained. Compared to the wild-type gene editing protein, the gene editing protein variant of the present invention can have cis-cleavage activity and reduced trans-cleavage activity, or even no trans-cleavage activity. Moreover, the gene editing protein variant of the present invention and the gene editing system comprising the gene editing protein variant of the present invention can significantly reduce the gene editing off-target rate. On this basis, the inventors have completed the present invention.

### Terms

To facilitate a better understanding of the present disclosure, certain terms are first defined. As used in the present application, unless otherwise expressly defined herein, each of the following terms shall have the meaning given below. Other definitions are expounded throughout the application.

The term “about” may refer to a value or composition within an acceptable error range of a particular value or composition determined by a person of ordinary skill in the art, which will partly depend on how the value or composition is measured or determined. For example, as used herein, the expression “about 100” includes all values between 99 and 101 (such as 99.1, 99.2, 99.3, 99.4, etc.).

As used herein, the term “contain” or “comprise (or include)” can be open, semi-closed, and closed. In other words, the terms also include “substantially consisting of” or “consisting of”.

Sequence identity (or homology) is determined by comparing two aligned sequences along a predetermined comparison window (it can be 50%, 60%, 70%, 80%, 90%, 95% or 100% of the length of the reference nucleotide sequence or protein) and by determining the number of positions where the same residue appears. Usually, this is represented as a percentage. The measurement of sequence identity of nucleotide sequences is a method well known to those skilled in the art.

### Cis-Cleavage Activity

In the present invention, cis-cleavage activity refers to the specific cleavage activity of Cas proteins on target nucleic acid molecules.

### Trans-Cleavage Activity

In the present invention, trans-cleavage activity refers to the non-specific cleavage activity of Cas proteins on non-target nucleic acid molecules (mainly non-target single-stranded nucleic acid molecules).

When DNAs are in a state of replication or transcription, the double-stranded DNAs will unwind into single-stranded DNAs. At this point, the trans-cleavage activity of gene editing proteins (such as Cas12a) may lead to the cleavage of these single-stranded DNAs, resulting in off-target cleavage. Therefore, reducing the trans-cleavage activity of gene editing proteins is equivalent to reducing the gene editing off-target rate of gene editing proteins.

### Wild-Type Gene Editing Protein

As used herein, “wild-type gene editing protein” refers to a naturally occurring, unmodified gene editing protein, the nucleotide of which can be obtained through genetic engineering techniques such as genome sequencing, polymerase chain reaction (PCR), etc., and its amino acid sequence can be deduced from the nucleotide sequence. The origin of the wild-type gene editing protein comprises Lachnospiraceae bacterium ND2006 (LbCas12a), Thiomicrospira sp. XS5 (TsCas12a), Francisella tularensis (FnCas12a), Bacteroidetes oral taxon 274 (BoCas12a), Oribacterium sp. NK2B42 (OsCas12a), Acidaminococcus sp. BV3L6 (AsCas12a), Helcococcus sp kunzii (HkCas12a), and Lachnospiraceae bacterium NC2008 (Lb5Cas12a). The wild-type gene editing protein comprises Cas12, Cas14, and further comprises Cas12a, Cas12b, Cas12e; furthermore, the Cas12a is selected from the group consisting of: FnCas12a, LbCas12a, ErCas12a, Evcas12a, Lb5Cas12a, HkCas12a, OsCas12a, TsCas12a, BbCas12a, BoCas12a, Lb4Cas12a, CeCas12a, PrCas12a, CsbCas12a, BhCas12a, SsCas12a, Lb3Cas12a, BpCas12a, PdCas12a, BfCas12a, PcCas12a, cMtCas12a, PeCas12a, LiCas12a, Lb2Cas12a, PmCas12a, MbCas12a, EeCas12a, CsbCas12a, ArCas12a, BsCas12a, AbCas12a, AsCas12a, and a combination thereof.

In a preferred embodiment of the present invention, the wild-type gene editing protein is FnCas12a, whose sequence is shown in SEQ ID NO.1.

Wild-type FnCas12a amino acid sequence (SEQ ID NO. 1):
MSIYQEFVNKYSLSKTLRFELIPQGKTLENIKARGLILDDEKRAKDYKKAKQIIDKY
HQFFIEEILSSVCISEDLLQNYSDVYFKLKKSDDDNLQKDFKSAKDTIKKQISEYIKDSEK
FKNLFNQNLIDAKKGQESDLILWLKQSKDNGIELFKANSDITDIDEALEIIKSFKGWTTYF
KGFHENRKNVYSSNDIPTSIIYRIVDDNLPKFLENKAKYESLKDKAPEAINYEQIKKDLAE
ELTFDIDYKTSEVNQRVFSLDEVFEIANFNNYLNQSGITKFNTIIGGKFVNGENTKRKGIN
EYINLYSQQINDKTLKKYKMSVLFKQILSDTESKSFVIDKLEDDSDVVTTMQSFYEQIAA
FKTVEEKSIKETLSLLFDDLKAQKLDLSKIYFKNDKSLTDLSQQVEDDYSVIGTAVLEYIT
QQIAPKNLDNPSKEQELIAKKTEKAKYLSLETIKLALEEFNKHRDIDKQCRFEEILANFAA
IPMIFDEIAQNKDNLAQISIKYQNQGKKDLLQASAEDDVKAIKDLLDQTNNLLHKLKIFH
ISQSEDKANILDKDEHFYLVFEECYFELANIVPLYNKIRNYITQKPYSDEKFKLNFENSTL
ANGWDKNKEPDNTAILFIKDDKYYLGVMNKKNNKIFDDKAIKENKGEGYKKIVYKLLP
GANKMLPKVFFSAKSIKFYNPSEDILRIRNHSTHTKNGSPQKGYEKFEFNIEDCRKFIDFY
KQSISKHPEWKDFGFRFSDTQRYNSIDEFYREVENQGYKLTFENISESYIDSVVNQGKLY
LFQIYNKDFSAYSKGRPNLHTLYWKALFDERNLQDVVYKLNGEAELFYRKQSIPKKITH
PAKEAIANKNKDNPKKESVFEYDLIKDKRFTEDKFFFHCPITINFKSSGANKFNDEINLLL
KEKANDVHILSIDRGERHLAYYTLVDGKGNIIKQDTFNIIGNDRMKTNYHDKLAAIEKD
RDSARKDWKKINNIKEMKEGYLSQVVHEIAKLVIEYNAIVVFEDLNFGFKRGRFKVEKQ
VYQKLEKMLIEKLNYLVFKDNEFDKTGGVLRAYQLTAPFETFKKMGKQTGIIYYVPAGF
TSKICPVTGFVNQLYPKYESVSKSQEFFSKFDKICYNLDKGYFEFSFDYKNFGDKAAKG
KWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANG
AYHIGLKGLMLLGRIKNNQEGKKLNLVIKNEEYFEFVQNRNN*

### Gene Editing Protein Variant and Encoding Nucleic Acids Thereof

As used herein, the terms “gene editing protein variant”, “variant of the present invention”, “gene editing mutant protein of the present invention”, and “mutant protein” can be used interchangeably, all referring to non-naturally occurring mutated gene editing proteins with cis-cleavage activity, and wherein the mutant protein undergoes mutation in the wild-type gene editing protein at one or more cleavage activity-related core amino acid sites selected from the group consisting of:
a phenylalanine (F) site corresponding to the 1081st position of FnCas12a; and/or
a lysine (K) site corresponding to the 1069th position of FnCas12a, and the mutant protein has reduced trans-cleavage activity, or even no trans-cleavage activity as compared to the wild-type gene editing protein thereof.

The term “core amino acid” refers to a specific amino acid as described herein at the corresponding position in a sequence that is based on a wild-type gene editing protein and has least 80% homology with the wild-type gene editing protein, such as 84%, 85%, 90%, 92%, 95%, 98%, or 99%. For example, based on the wild-type gene editing protein, the core amino acid is:
a phenylalanine (F) corresponding to the 1081st position of FnCas12a; and/or
a lysine (K) corresponding to the 1069th position of FnCas12a.

In addition, the mutant protein obtained by mutating the core amino acids mentioned above has cis-cleavage activity and reduced trans-cleavage activity, or even no trans-cleavage activity.

Preferably, in the present invention, the core amino acids of the present invention undergo the following mutations:
the phenylalanine (F) at the 1081st position of FnCas12a is mutated to arginine (R);
the lysine (K) at the 1069th position of FnCas12a is mutated to arginine (R).

It should be understood that the amino acid numbering in the mutant protein of the present invention is based on the wild-type gene editing protein. When a specific mutant protein has 80% or more homology with the sequence of the wild-type gene editing protein, there may be a misalignment in the amino acid numbering of the mutant protein relative to that of the wild-type gene editing protein, such as a misalignment of positions 1-100 to the N-terminus or C-terminus of the amino acid. By using conventional sequence alignment techniques in the art, those skilled in the art can generally understand that such misalignment is within a reasonable range, and that mutant proteins with 80% (such as 90%, 95%, 98%) homology, which have the same or similar cis-cleavage activity and reduced trans-cleavage activity, should not be excluded from the scope of the mutant protein of the present invention due to the misalignment in amino acid numbering.

The mutant protein of the present invention is a synthetic protein or a recombinant protein, that is, it can be a chemically synthesized product, or produced from a prokaryotic or eukaryotic host (for example, bacteria, yeast, and plants) using recombinant technology. Depending on the host used in the recombinant production protocol, the mutant protein of the present invention may be glycosylated or non-glycosylated. The mutant protein of the present invention may also include or not include the initial methionine residue.

The present invention also includes fragments, derivatives and analogs of the mutant protein. As used herein, the terms “fragment”, “derivative” and “analog” refer to a protein that substantially retains the same biological function or activity as the mutant protein.

The mutant protein fragment, derivative or analog of the present invention may be (i) a mutant protein in which one or more conserved or non-conserved amino acid residues (preferably conserved amino acid residues) are substituted, and such substituted amino acid residues may or may not be encoded by the genetic code, or (ii) a mutant protein with a substitution group in one or more amino acid residues, or (iii) a mutant protein formed by fusion of a mature mutant protein with another compound (such as a compound that prolongs the half-life of the mutant protein, such as polyethylene glycol), or (iv) a mutant protein formed by fusion of an additional amino acid sequence to this mutant protein sequence (such as a leader sequence or secretory sequence or sequence used to purify this mutant protein or proprotein sequence, or a fusion protein formed with an antigen IgG fragment). According to the teachings herein, these fragments, derivatives and analogs are within the scope known to those skilled in the art. In the present invention, the amino acids subjected to conserved substitution are preferably generated by amino acid substitutions according to Table I.

TABLE I

| Initial residue | Representative substitution | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

The active mutant protein of the present invention has cis-cleavage activity and reduced trans-cleavage activity, or even no trans-cleavage activity.

Preferably, the mutant protein is as shown in any one of SEQ ID NOs.: 2-3.

Mutant protein FnCas12a^{K1069R} amino acid sequence:
(SEQ ID NO. 2)
MSIYQEFVNKYSLSKTLRFELIPQGKTLENIKARGLILDDEKRAKDYKKAKQIIDKY
HQFFIEEILSSVCISEDLLQNYSDVYFKLKKSDDDNLQKDFKSAKDTIKKQISEYIKDSEK
FKNLFNQNLIDAKKGQESDLILWLKQSKDNGIELFKANSDITDIDEALEIIKSFKGWTTYF
KGFHENRKNVYSSNDIPTSIIYRIVDDNLPKFLENKAKYESLKDKAPEAINYEQIKKDLAE
ELTFDIDYKTSEVNQRVFSLDEVFEIANFNNYLNQSGITKFNTIIGGKFVNGENTKRKGIN
EYINLYSQQINDKTLKKYKMSVLFKQILSDTESKSFVIDKLEDDSDVVTTMQSFYEQIAA
FKTVEEKSIKETLSLLFDDLKAQKLDLSKIYFKNDKSLTDLSQQVEDDYSVIGTAVLEYIT
QQIAPKNLDNPSKEQELIAKKTEKAKYLSLETIKLALEEFNKHRDIDKQCRFEEILANFAA
IPMIFDEIAQNKDNLAQISIKYQNQGKKDLLQASAEDDVKAIKDLLDQTNNLLHKLKIFH
ISQSEDKANILDKDEHFYLVFEECYFELANIVPLYNKIRNYITQKPYSDEKFKLNFENSTL
ANGWDKNKEPDNTAILFIKDDKYYLGVMNKKNNKIFDDKAIKENKGEGYKKIVYKLLP
GANKMLPKVFFSAKSIKFYNPSEDILRIRNHSTHTKNGSPQKGYEKFEFNIEDCRKFIDFY
KQSISKHPEWKDFGFRFSDTQRYNSIDEFYREVENQGYKLTFENISESYIDSVVNQGKLY
LFQIYNKDFSAYSKGRPNLHTLYWKALFDERNLQDVVYKLNGEAELFYRKQSIPKKITH
PAKEAIANKNKDNPKKESVFEYDLIKDKRFTEDKFFFHCPITINFKSSGANKFNDEINLLL
KEKANDVHILSIDRGERHLAYYTLVDGKGNIIKQDTFNIIGNDRMKTNYHDKLAAIEKD
RDSARKDWKKINNIKEMKEGYLSQVVHEIAKLVIEYNAIVVFEDLNFGFKRGRFKVEKQ
VYQKLEKMLIEKLNYLVFKDNEFDKTGGVLRAYQLTAPFETFKKMGRQTGIIYYVPAGF
TSKICPVTGFVNQLYPKYESVSKSQEFFSKFDKICYNLDKGYFEFSFDYKNFGDKAAKG
KWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANG
AYHIGLKGLMLLGRIKNNQEGKKLNLVIKNEEYFEFVQNRNN*
Mutant protein FnCas12a^{F1081R} amino acid sequence:
(SEQ ID NO. 3)
MSIYQEFVNKYSLSKTLRFELIPQGKTLENIKARGLILDDEKRAKDYKKAKQIIDKY
HQFFIEEILSSVCISEDLLQNYSDVYFKLKKSDDDNLQKDFKSAKDTIKKQISEYIKDSEK
FKNLFNQNLIDAKKGQESDLILWLKQSKDNGIELFKANSDITDIDEALEIIKSFKGWTTYF
KGFHENRKNVYSSNDIPTSIIYRIVDDNLPKFLENKAKYESLKDKAPEAINYEQIKKDLAE
ELTFDIDYKTSEVNQRVFSLDEVFEIANFNNYLNQSGITKFNTIIGGKFVNGENTKRKGIN
EYINLYSQQINDKTLKKYKMSVLFKQILSDTESKSFVIDKLEDDSDVVTTMQSFYEQIAA
FKTVEEKSIKETLSLLFDDLKAQKLDLSKIYFKNDKSLTDLSQQVEDDYSVIGTAVLEYIT
QQIAPKNLDNPSKEQELIAKKTEKAKYLSLETIKLALEEFNKHRDIDKQCRFEEILANFAA
IPMIFDEIAQNKDNLAQISIKYQNQGKKDLLQASAEDDVKAIKDLLDQTNNLLHKLKIFH
ISQSEDKANILDKDEHFYLVFEECYFELANIVPLYNKIRNYITQKPYSDEKFKLNFENSTL
ANGWDKNKEPDNTAILFIKDDKYYLGVMNKKNNKIFDDKAIKENKGEGYKKIVYKLLP
GANKMLPKVFFSAKSIKFYNPSEDILRIRNHSTHTKNGSPQKGYEKFEFNIEDCRKFIDFY
KQSISKHPEWKDFGFRFSDTQRYNSIDEFYREVENQGYKLTFENISESYIDSVVNQGKLY
LFQIYNKDFSAYSKGRPNLHTLYWKALFDERNLQDVVYKLNGEAELFYRKQSIPKKITH
PAKEAIANKNKDNPKKESVFEYDLIKDKRFTEDKFFFHCPITINFKSSGANKFNDEINLLL
KEKANDVHILSIDRGERHLAYYTLVDGKGNIIKQDTFNIIGNDRMKTNYHDKLAAIEKD
RDSARKDWKKINNIKEMKEGYLSQVVHEIAKLVIEYNAIVVFEDLNFGFKRGRFKVEKQ
VYQKLEKMLIEKLNYLVFKDNEFDKTGGVLRAYQLTAPFETFKKMGKQTGIIYYVPAG
RTSKICPVTGFVNQLYPKYESVSKSQEFFSKFDKICYNLDKGYFEFSFDYKNFGDKAAKG
KWTIASFGSRLINFRNSDKNHNWDTREVYPTKELEKLLKDYSIEYGHGECIKAAICGESD
KKFFAKLTSVLNTILQMRNSKTGTELDYLISPVADVNGNFFDSRQAPKNMPQDADANG
AYHIGLKGLMLLGRIKNNQEGKKLNLVIKNEEYFEFVQNRNN*

It should be understood that, compared with the sequence as shown in any one of SEQ ID NOs.: 2-3, the mutant protein of the present invention generally has a high homology (or identity). Preferably, the homology of the mutant protein with the sequence as shown in any one of SEQ ID NOs.: 2-3 is at least 80%, preferably at least 85%-90%, more preferably at least 95%, and most preferably at least 98% or 99%.

In addition, the mutant protein of the present invention can also be modified. Modification (typically without altering the primary structure) forms include: chemically derived forms of the mutant protein in vivo or in vitro, such as acetylation or carboxylation. Modifications also include glycosylation, such as those mutant proteins produced by glycosylation modification during the synthesis and processing of the mutant protein, or during further processing steps. This modification can be accomplished by exposing the mutant protein to an enzyme that catalyzes glycosylation (such as a mammalian glycosylase or deglycosylase). Modification forms also include sequences having phosphorylated amino acid residues (such as phosphotyrosine, phosphoserine, phosphothreonine). Mutant proteins modified to enhance their resistance to protein hydrolysis or to optimize their solubility are also included.

The term “polynucleotide encoding the mutant protein” may include polynucleotides encoding the mutant protein of the present invention, and may also include polynucleotides with additional coding and/or non-coding sequences.

In a preferred embodiment, the sequence of the polynucleotide encoding the mutant protein of the present invention is as shown in any one of SEQ ID NOs.: 4-5.

FnCas12a^{K1069R} nucleotide sequence (SEQ ID NO. 4):
atgagcatctatcaggagttcgtgaataagtacagcctgtccaagaccctgcggtttgagctgatcccccagggcaagacactgg
agaacatcaaggccaggggcctgatcctggacgatgagaagcgcgccaaggactataagaaggccaagcagatcatcgataagtacc
accagttctttatcgaggagatcctgagcagcgtgtgcatctctgaggatctgctgcagaattacagcgacgtgtatttcaagctgaagaa
gtctgacgatgacaacctgcagaaggacttcaagagcgccaaggacaccatcaagaagcagatcagcgagtatatcaaggactccga
gaagtttaagaatctgttcaaccagaatctgatcgatgccaagaagggccaggagtccgacctgatcctgtggctgaagcagtctaagga
caatggcatcgagctgttcaaggccaactctgatatcaccgatatcgacgaggccctggagatcatcaagagctttaagggctggaccac
atactttaagggcttccacgagaacaggaagaacgtgtacagcagcaacgacatccctacaagcatcatctaccgcatcgtggatgaca
atctgccaaagttcctggagaacaaggccaagtatgagtccctgaaggacaaggcccccgaggccatcaattacgagcagatcaagaa
ggatctggccgaggagctgaccttcgatatcgactataagacatccgaggtgaaccagcgggtgttttctctggacgaggtgtttgagatc
gccaatttcaacaattacctgaaccagtccggcatcaccaagttcaatacaatcatcggcggcaagtttgtgaacggcgagaataccaag
agaaagggcatcaacgagtacatcaatctgtatagccagcagatcaacgacaagaccctgaagaagtacaagatgagcgtgctgttcaa
gcagatcctgtccgatacagagtctaagagctttgtgatcgataagctggaggatgactctgacgtggtgaccacaatgcagagcttttat
gagcagatcgccgccttcaagaccgtggaggagaagtctatcaaggagacactgagcctgctgttcgatgacctgaaggcccagaagc
tggacctgtctaagatctacttcaagaacgataagtccctgaccgacctgtctcagcaggtgtttgatgactatagcgtgatcggcaccgc
cgtgctggagtacatcacacagcagatcgccccaaagaacctggataatccctctaagaaggagcaggagctgatcgccaagaagac
cgagaaggccaagtatctgagcctggagacaatcaagctggccctggaggagttcaataagcaccgggatatcgacaagcagtgcag
atttgaggagatcctggccaacttcgccgccatccccatgatctttgatgagatcgcccagaacaaggacaatctggcccagatctccatc
aagtaccagaaccagggcaagaaggacctgctgcaggcctctgccgaggatgacgtgaaggccatcaaggatctgctggaccagacc
aacaatctgctgcacaagctgaagatcttccacatctcccagtctgaggataaggccaatatcctggataaggacgagcacttttatctggt
gttcgaggagtgttacttcgagctggccaacatcgtgcccctgtacaacaagatcagaaattatatcacacagaagccttactccgacgag
aagtttaagctgaacttcgagaacagcaccctggccaacggctgggataagaataaggagcctgacaacacagccatcctgttcatcaa
ggatgacaagtactatctgggcgtgatgaataagaagaacaataagatcttcgatgacaaggccatcaaggagaacaagggcgagggc
tacaagaagatcgtgtataagctgctgcccggcgccaataagatgctgcctaaggtgttcttttccgccaagtctatcaagttctacaaccc
atccgaggacatcctgcggatcagaaatcactccacccacacaaagaacggctctccccagaagggctatgagaagtttgagttcaatat
cgaggattgccggaagtttatcgacttctacaagcagagcatctccaagcaccctgagtggaaggattttggcttcaggtttagcgacacc
cagcggtacaactccatcgacgagttctacagagaggtggagaatcagggctataagctgacatttgagaacatctctgagagctacatc
gacagcgtggtgaatcagggcaagctgtacctgttccagatctataacaaggacttcagcgcctattccaagggccggccaaacctgca
caccctgtactggaaggccctgttcgatgagagaaatctgcaggacgtggtgtataagctgaacggcgaggccgagctgttttacagga
agcagtccatccctaagaagatcacacacccagccaaggaggccatcgccaacaagaataaggacaatcctaagaaggagagcgtgt
tcgagtacgatctgatcaaggacaagcggttcaccgaggataagttctttttccactgtccaatcacaatcaacttcaagtcctctggcgcc
aacaagtttaatgacgagatcaatctgctgctgaaggagaaggccaacgatgtgcacatcctgagcatcgaccggggcgagagacacc
tggcctactataccctggtggatggcaagggcaatatcatcaagcaggataccttcaacatcatcggcaatgacaggatgaagacaaact
accacgataagctggccgccatcgagaaggatagggactccgcccgcaaggactggaagaagatcaacaatatcaaggagatgaag
gagggctatctgtctcaggtggtgcacgagatcgccaagctggtcatcgagtacaatgccatcgtggtgttcgaggatctgaacttcggct
ttaagaggggccgctttaaggtggagaagcaggtgtatcagaagctggagaagatgctgatcgagaagctgaattacctggtgtttaag
gataacgagttcgacaagaccggaggcgtgctgagggcataccagctgaccgccccctttgagacattcaagaagatgggcAGgca
gacaggcatcatctactatgtgccagccggcttcacctccaagatctgccccgtgacaggctttgtgaaccagctgtaccctaagtatgag
tccgtgtctaagagccaggagtttttcagcaagttcgataagatctgttataatctggacaagggctacttcgagttttccttcgattataaga
actttggcgacaaggccgccaagggcaagtggaccatcgcctctttcggcagccggctgatcaactttagaaattccgataagaaccac
aattgggacacccgggaggtgtacccaacaaaggagctggagaagctgctgaaggactacagcatcgagtatggccacggcgagtgc
atcaaggccgccatctgtggcgagagcgataagaagtttttcgccaagctgacctccgtgctgaatacaatcctgcagatgcggaacag
caagaccggcacagagctggactacctgatctcccccgtggccgatgtgaacggcaacttcttcgacagcagacaggcccccaagaat
atgcctcaggatgccgacgccaacggcgcctatcacatcggcctgaagggcctgatgctgctgggcaggatcaagaacaatcaggag
ggcaagaagctgaacctggtcatcaagaacgaggagtactttgagttcgtgcagaaccgcaacaattga
FnCas12a^{F1081R} nucleotide sequence (SEQ ID NO. 5):
atgagcatctatcaggagttcgtgaataagtacagcctgtccaagaccctgcggtttgagctgatcccccagggcaagacactgg
agaacatcaaggccaggggcctgatcctggacgatgagaagcgcgccaaggactataagaaggccaagcagatcatcgataagtacc
accagttctttatcgaggagatcctgagcagcgtgtgcatctctgaggatctgctgcagaattacagcgacgtgtatttcaagctgaagaa
gtctgacgatgacaacctgcagaaggacttcaagagcgccaaggacaccatcaagaagcagatcagcgagtatatcaaggactccga
gaagtttaagaatctgttcaaccagaatctgatcgatgccaagaagggccaggagtccgacctgatcctgtggctgaagcagtctaagga
caatggcatcgagctgttcaaggccaactctgatatcaccgatatcgacgaggccctggagatcatcaagagctttaagggctggaccac
atactttaagggcttccacgagaacaggaagaacgtgtacagcagcaacgacatccctacaagcatcatctaccgcatcgtggatgaca
atctgccaaagttcctggagaacaaggccaagtatgagtccctgaaggacaaggcccccgaggccatcaattacgagcagatcaagaa
ggatctggccgaggagctgaccttcgatatcgactataagacatccgaggtgaaccagcgggtgttttctctggacgaggtgtttgagatc
gccaatttcaacaattacctgaaccagtccggcatcaccaagttcaatacaatcatcggcggcaagtttgtgaacggcgagaataccaag
agaaagggcatcaacgagtacatcaatctgtatagccagcagatcaacgacaagaccctgaagaagtacaagatgagcgtgctgttcaa
gcagatcctgtccgatacagagtctaagagctttgtgatcgataagctggaggatgactctgacgtggtgaccacaatgcagagcttttat
gagcagatcgccgccttcaagaccgtggaggagaagtctatcaaggagacactgagcctgctgttcgatgacctgaaggcccagaagc
tggacctgtctaagatctacttcaagaacgataagtccctgaccgacctgtctcagcaggtgtttgatgactatagcgtgatcggcaccgc
cgtgctggagtacatcacacagcagatcgccccaaagaacctggataatccctctaagaaggagcaggagctgatcgccaagaagac
cgagaaggccaagtatctgagcctggagacaatcaagctggccctggaggagttcaataagcaccgggatatcgacaagcagtgcag
atttgaggagatcctggccaacttcgccgccatccccatgatctttgatgagatcgcccagaacaaggacaatctggcccagatctccatc
aagtaccagaaccagggcaagaaggacctgctgcaggcctctgccgaggatgacgtgaaggccatcaaggatctgctggaccagacc
aacaatctgctgcacaagctgaagatcttccacatctcccagtctgaggataaggccaatatcctggataaggacgagcacttttatctggt
gttcgaggagtgttacttcgagctggccaacatcgtgcccctgtacaacaagatcagaaattatatcacacagaagccttactccgacgag
aagtttaagctgaacttcgagaacagcaccctggccaacggctgggataagaataaggagcctgacaacacagccatcctgttcatcaa
ggatgacaagtactatctgggcgtgatgaataagaagaacaataagatcttcgatgacaaggccatcaaggagaacaagggcgagggc
tacaagaagatcgtgtataagctgctgcccggcgccaataagatgctgcctaaggtgttcttttccgccaagtctatcaagttctacaaccc
atccgaggacatcctgcggatcagaaatcactccacccacacaaagaacggctctccccagaagggctatgagaagtttgagttcaatat
cgaggattgccggaagtttatcgacttctacaagcagagcatctccaagcaccctgagtggaaggattttggcttcaggtttagcgacacc
cagcggtacaactccatcgacgagttctacagagaggtggagaatcagggctataagctgacatttgagaacatctctgagagctacatc
gacagcgtggtgaatcagggcaagctgtacctgttccagatctataacaaggacttcagcgcctattccaagggccggccaaacctgca
caccctgtactggaaggccctgttcgatgagagaaatctgcaggacgtggtgtataagctgaacggcgaggccgagctgttttacagga
agcagtccatccctaagaagatcacacacccagccaaggaggccatcgccaacaagaataaggacaatcctaagaaggagagcgtgt
tcgagtacgatctgatcaaggacaagcggttcaccgaggataagttctttttccactgtccaatcacaatcaacttcaagtcctctggcgcc
aacaagtttaatgacgagatcaatctgctgctgaaggagaaggccaacgatgtgcacatcctgagcatcgaccggggcgagagacacc
tggcctactataccctggtggatggcaagggcaatatcatcaagcaggataccttcaacatcatcggcaatgacaggatgaagacaaact
accacgataagctggccgccatcgagaaggatagggactccgcccgcaaggactggaagaagatcaacaatatcaaggagatgaag
gagggctatctgtctcaggtggtgcacgagatcgccaagctggtcatcgagtacaatgccatcgtggtgttcgaggatctgaacttcggct
ttaagaggggccgctttaaggtggagaagcaggtgtatcagaagctggagaagatgctgatcgagaagctgaattacctggtgtttaag
gataacgagttcgacaagaccggaggcgtgctgagggcataccagctgaccgccccctttgagacattcaagaagatgggcaagcag
acaggcatcatctactatgtgccagccggcCGcacctccaagatctgccccgtgacaggctttgtgaaccagctgtaccctaagtatga
gtccgtgtctaagagccaggagtttttcagcaagttcgataagatctgttataatctggacaagggctacttcgagttttccttcgattataag
aactttggcgacaaggccgccaagggcaagtggaccatcgcctctttcggcagccggctgatcaactttagaaattccgataagaacca
caattgggacacccgggaggtgtacccaacaaaggagctggagaagctgctgaaggactacagcatcgagtatggccacggcgagtg
catcaaggccgccatctgtggcgagagcgataagaagtttttcgccaagctgacctccgtgctgaatacaatcctgcagatgcggaaca
gcaagaccggcacagagctggactacctgatctcccccgtggccgatgtgaacggcaacttcttcgacagcagacaggcccccaaga
atatgcctcaggatgccgacgccaacggcgcctatcacatcggcctgaagggcctgatgctgctgggcaggatcaagaacaatcagga
gggcaagaagctgaacctggtcatcaagaacgaggagtactttgagttcgtgcagaaccgcaacaattga

The present invention also relates to variants of the aforementioned polynucleotides, which encode fragments, analogs, and derivatives of polypeptides or mutant proteins having the same amino acid sequence as those of the present invention. These nucleotide variants include substitution variants, deletion variants, and insertion variants. As known in the art, an allelic variant is an alternative form of a polynucleotide that may involve substitution, deletion, or insertion of one or more nucleotides, but does not substantially alter the function of the mutant protein it encodes.

The present invention further relates to polynucleotides that hybridize with the aforementioned sequences and have at least 50% identity, preferably at least 70% identity, and more preferably at least 80% identity between the two sequences. The present invention particularly concerns polynucleotides that can hybridize with the polynucleotide of the present invention under strict conditions (or stringent conditions). In the present invention, “strict conditions” refer to: (1) hybridization and elution at lower ionic strength and higher temperature, such as 0.2×SSC, 0.1% SDS, 60° C.; or (2) hybridization in the presence of denaturants, such as 50% (v/v) formamide, 0.1% calf serum/0.1% Ficoll, 42° C., etc.; or (3) hybridization occurring only when the identity between the two sequences is at least 90% or more, preferably 95% or more.

The mutant protein and polynucleotide of the present invention are preferably provided in an isolated form, and preferably, are purified to homogeneity.

The full-length sequence of the polynucleotide of the present invention can generally be obtained through PCR amplification method, recombinant method, or artificial synthesis method. For PCR amplification method, primers can be designed based on the related nucleotide sequences disclosed in the present invention, especially the open reading frame sequences, and commercially available cDNA libraries or cDNA libraries prepared by conventional methods known to those skilled in the art can be used as templates to amplify the related sequences. When the sequence is long, it often requires two or more rounds of PCR amplification, followed by assembly of the amplified fragments in the correct order.

Once the related sequences are obtained, they can be obtained in large quantities using the recombinant method. This typically involves cloning them into vectors, transferring them into cells, and then isolating and obtaining the related sequences from the proliferated host cells using conventional methods.

In addition, related sequences can also be synthesized through the artificial synthesis method, especially when the fragment length is short. Generally, long sequence fragments can be obtained by synthesizing multiple small fragments first and then ligating them together.

Currently, DNA sequences encoding the proteins (or fragments thereof, or derivatives thereof) of the present invention can be completely obtained through chemical synthesis. The DNA sequences can then be introduced into various existing DNA molecules (or such as vectors) and cells known in the art. In addition, mutations can be introduced into the sequence of the protein of the present invention through chemical synthesis.

The method of amplifying DNA/RNA using PCR technology is preferably used to obtain the polynucleotide of the present invention. Especially when it is difficult to obtain full-length cDNAs from libraries, the RACE method (RACE-cDNA end rapid amplification method) can be preferably used. Primers for PCR can be appropriately selected based on the sequence information of the present invention disclosed herein and synthesized using conventional methods. The amplified DNA/RNA fragments can be isolated and purified by conventional methods, such as gel electrophoresis.

It should be noted that the 1081st and 1069th positions in the amino acid sequence of the gene editing protein (FnCas12a) derived from Francisella tularensis in the present invention correspond to conserved positions in Cas12a from other origins. The specific correspondence is shown in Table II.

TABLE II
Mutated Amino Acid Corresponding Sites

| NCBI Sequence | | | |
|---|---|---|---|
| Accession | | Corresponding | Corresponding |
| Number | Protein Type | Mutation Site 1 | Mutation Site 2 |
| 489130501 | FnCas12a | K1069 | F1081 |
| 987324269 | BbCas12a | K1007 | N1019 |
| 545612232 | AsCas12a | T1057 | Y1069 |
| 496509559 | BoCas12a | K1021 | N1033 |
| 491540987 | HkCas12a | G1078 | Y1090 |
| 769130406 | Lb4Cas12a | R992 | N1004 |
| 652820612 | Lb5Cas12a | K968 | L980 |
| 917059416 | LbCas12a | T1006 | L1018 |
| 909652572 | OsCas12a | K989 | L1001 |
| 972924080 | TsCas12a | K1058 | Y1070 |

Therefore, mutations at the aforementioned sites play a crucial role in reducing the gene editing off-target rate.

### Expression Vector and Host Cell

The present invention also relates to a vector comprising the polynucleotide of the present invention, as well as a host cell genetically engineered with the vector of the present invention or with the coding sequence of the mutant protein of the present invention, and a method for producing the polypeptide of the present invention through recombinant techniques.

Using conventional recombinant DNA technology, the polynucleotide sequence of the present invention can be utilized to express or produce a recombinant mutant protein. Generally, the following steps are involved:
(1) Transforming or transfecting a suitable host cell with the polynucleotide (or variant) of the present invention encoding the mutant protein of the present invention, or with a recombinant expression vector comprising this polynucleotide;
(2) Culturing the host cell in a suitable medium;
(3) Isolating and purifying the protein from the medium or cell.

In the present invention, the polynucleotide sequence encoding the mutant protein can be inserted into a recombinant expression vector. The term “recombinant expression vector” refers to bacterial plasmids, bacteriophages, yeast plasmids, plant cell viruses, mammalian cell viruses such as adenoviruses, retroviruses, or other vectors well-known in the art. Any plasmids and vectors can be used as long as they can replicate and remain stable within the hosts. An important feature of the expression vector is that it typically contains an origin of replication, a promoter, a marker gene, and a translation control element.

Methods well-known to those skilled in the art can be used to construct an expression vector which comprises a DNA sequence encoding the mutant protein of the present invention and appropriate transcription/translation control signals. These methods include in vitro recombinant DNA technology, DNA synthesis technology, in vivo recombinant technology, etc. The DNA sequence can be effectively linked to an appropriate promoter in the expression vector to guide mRNA synthesis. Representative examples of these promoters include: the lac or trp promoter of Escherichia coli; the λ phage PL promoter; eukaryotic promoters comprising the CMV immediate-early promoter, HSV thymidine kinase promoter, early and late SV40 promoter, LTRs of retroviruses; and some other known promoters that can control gene expression in prokaryotic or eukaryotic cells or viruses thereof. The expression vector also includes a ribosome binding site for translation initiation and a transcription terminator.

Furthermore, the expression vector preferably comprises one or more selectable marker genes to provide phenotypic traits for selecting transformed host cells, such as dihydrofolate reductase, neomycin resistance, and green fluorescent protein (GFP) for eukaryotic cell culture, or tetracycline or ampicillin resistance for Escherichia coli.

Vectors comprising the appropriate DNA sequences and appropriate promoters or control sequences described above can be used to transform appropriate host cells to enable them to express the proteins.

Host cells can be prokaryotic cells (such as Escherichia coli), or lower eukaryotic cells, or higher eukaryotic cells, such as yeast cells, plant cells, or mammalian cells (including humans and non-human mammals). Representative examples comprise: Escherichia coli, wheat germ cells, insect cells, SF9, HeLa, HEK293, CHO, yeast cells, etc. In a preferred embodiment of the present invention, yeast cells (such as Pichia pastoris, Kluyveromyces sp., and a combination thereof; preferably, the yeast cells comprise: Kluyveromyces sp., more preferably Kluyveromyces marxianus, and/or Kluyveromyces lactis) are selected as host cells.

When the polynucleotide of the present invention is expressed in higher eukaryotic cells, if an enhancer sequence is inserted into the vector, the transcription will be enhanced. Enhancers are cis-acting factors of DNA, typically about 10 to 300 base pairs, which act on promoters to enhance gene transcription. Examples include the SV40 enhancer of 100 to 270 base pairs on the late side of the replication origin, the polyoma enhancer on the late side of the replication origin, and the adenovirus enhancer, etc.

Those skilled in the art generally know how to select appropriate vectors, promoters, enhancers, and host cells.

Transformation of host cells with recombinant DNA can be performed using conventional techniques well-known to those skilled in the art. When the host is a prokaryote such as Escherichia coli, competent cells capable of absorbing DNA can be harvested after the exponential growth phase and treated with the CaCl₂) method, with the steps being well-known in the art. Another method is to use MgCl₂. If needed, transformation can also be performed using electroporation method. When the host is a eukaryote, the following DNA transfection methods can be used: calcium phosphate coprecipitation, conventional mechanical methods such as microinjection, electroporation, liposome packaging, etc.

The obtained transformants can be cultured using conventional methods to express the polypeptide encoded by the gene of the present invention. Depending on the host cell used, the medium used for culture can be selected from various conventional media. Culturing is performed under conditions suitable for the growth of the host cells. After the host cells grow to an appropriate cell density, the selected promoter is induced using an appropriate method (such as temperature conversion or chemical induction), and the cells are cultured for an additional period.

The recombinant polypeptide in the above methods can be expressed within the cell, or on the cell membrane, or secreted outside the cell. If needed, the recombinant protein can be isolated and purified using various isolation methods based on its physical, chemical, and other properties. These methods are well-known to those skilled in the art. Examples of these methods include, but are not limited to: conventional renaturation treatment, treatment with protein precipitating agents (salting-out methods), centrifugation, osmotic lysis, ultra-treatment, ultracentrifugation, molecular sieve chromatography (gel filtration), adsorption chromatography, ion exchange chromatography, high-performance liquid chromatography (HPLC), and other various liquid chromatography techniques, as well as combinations of these methods.

### The Main Advantages of the Present Invention Include:

(1) The present invention first discovered a new gene editing protein variant. Compared to the wild-type gene editing protein, the gene editing protein variant of the present invention can have cis-cleavage activity and reduced trans-cleavage activity, or even no trans-cleavage activity. Moreover, the gene editing protein variant of the present invention and the gene editing system comprising the gene editing protein variant of the present invention can significantly reduce the gene editing off-target rate.

The present invention will be further illustrated below with reference to the specific examples. It should be understood that these examples are only to illustrate the present invention, not to limit the scope of the present invention. The conditions of the experimental methods not specifically indicated in the following examples are usually in accordance with conventional conditions as described in e.g., Sambrook et al., Molecular Cloning: A Laboratory Manual (New York: Cold Spring Harbor Laboratory Press, 1989), or according to the conditions recommended by the manufacturers. Percentages and parts are calculated by weight unless otherwise stated.

Unless otherwise stated, the reagents and materials used in the examples of the present invention are commercially available products.

### (I) Materials and Methods

### 1. FnCas12a Protein Mutation Experiment

### (1) Construction of FnCas12a Mutant Protein Expression Vector

Primers containing the mutation sites (sequences are shown in Table 1) were designed, and the wild-type FnCas12a expression plasmid was used as a template to amplify linear fragments having the desired site mutations using Phanta DNA polymerase. The amplified products were seamlessly ligated into a circular expression vector using Ezmax (obtained from Anhui Tolo Port Biotechnology Co., Ltd.). The reaction products were transformed into DH10B (obtained from Anhui Tolo Port Biotechnology Co., Ltd.), and the transformants were cultured overnight in LB medium containing 50 μg/mL Kan at 37° C. Single clones were picked and cultured in liquid LB medium containing 50 μg/mL Kan on a shaker at 37° C. overnight, and the plasmids were extracted. The correctly sequenced plasmids were stored at −80° C.

TABLE 1

| Mutation Site | Primer Name | Primer Sequence (5′ - 3′) |
|---|---|---|
| F1081R | F1081R-F | cacggggcagatcttggaggtgCGgccggctggcacatagtag |
| | | (SEQ ID NO. 6) |
| | F1081R-R | cacctccaagatctgccccgtgacaggc (SEQ ID NO. 7) |
| K1069R | K1069R-F | cttcttgaatgtctcaaagggggcggtcag (SEQ ID NO. 8) |
| | K1069R-R | cccctttgagacattcaagaagatgggcAGgcagacaggcatc |
| | | atctac (SEQ ID NO. 9) |

### (2) Purification of FnCas12a Mutant Protein

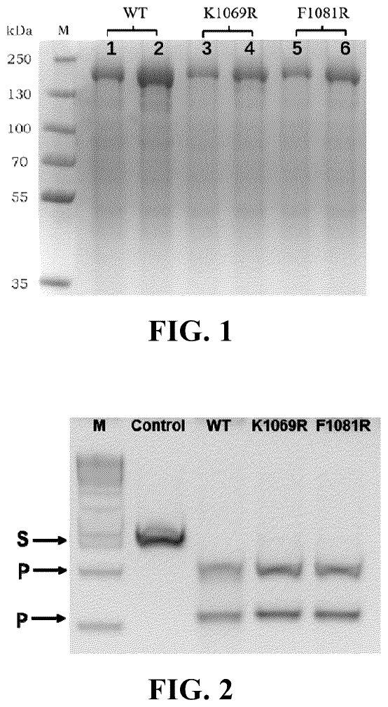

The constructed pET28TEV-FnCas12a plasmid was transformed into E. coli BL21 (DE3) competent cells, and the cells were cultured in solid LB medium containing 50 μg/mL kanamycin (hereinafter referred to as Kan) at 37° C. for 12-14 hours. Three single clones were picked and inoculated into 50 mL of liquid LB medium with Kan resistance, and after overnight cultivation at 37° C. on a shaker, they were transferred at a ratio of 1% (v/v) into 1 L of liquid LB medium with Kan resistance. The clones were cultured at 37° C. until the OD600 reached between 0.6-0.8, followed by ice bath for 30 minutes. IPTG was added to a final concentration of 0.2-0.5 mM, and the clones were cultured at 16° C. with 220 rpm for 14-16 hours. The culture was centrifuged at 16° C. and 6000 rpm for 5 minutes to collect the bacteria. The bacterial precipitate, after being weighed, was then subjected to osmotic lysis, or it can be temporarily stored at −80° C. (All the following steps should be performed at 4° C.). The bacterial precipitate was resuspended in a protein lysis buffer at a ratio of 5-10 mL per gram of bacteria, and PMSF protease inhibitor was added to a final concentration of 1 mM at the same time. After the bacteria were evenly resuspended, the resuspension solution was subjected to high-pressure lysis using a cell disruptor. The resulting lysis solution was centrifuged at 14000 rpm for 30 minutes, and the supernatant was collected. The protein supernatant obtained from centrifugation was mixed with Ni-NTA (Tiandi Renhe Biotechnology Co., Ltd.). The mixture was gently shaken at 4° C. for 1 hour to allow the protein to fully bind with the nickel column, and then it was loaded onto a 30 mL column. After the supernatant was drained, the column was washed with a wash buffer containing a low concentration of imidazole to remove impurity proteins. The target protein was eluted using an elution buffer containing a high concentration of imidazole, with the target protein being eluted in small volumes (for specific operating procedures, referring to the operating manual of Ni-NTA). The purity of the target protein was verified using a SDS-PAGE gel with a concentration of 10% (v/v). Several tubes of relatively pure target protein were combined, dialyzed overnight, and concentrated using a 50 kDa ultrafiltration tube. The purity of the protein is shown in FIG. 1. An equal volume of glycerol (pre-cooled to 4° C.) was mixed uniformly with the protein. The protein concentration was determined using the Bradford method. The protein was then aliquoted into small volumes and stored at −80° C., or can be stored at −20° C. for short-term use.

2. Preparation of Target dsDNA Sequence:

A PCR amplification was performed with AMED16s-F/R (Sequences: AMED16s-F: 5′-gtgaactaagccagtagagc-3′ (SEQ ID NO.10), AMED16s-R: 5′-ctttcgctcctcagcgtcag-3′ (SEQ ID NO.11), synthesized by Sangon Biotech (Shanghai) Co., Ltd.) as the amplification primers, and the Amycolatopsis mediterranei U32 genome (NCBI Accession Number: SAMN02603409) as the template. The PCR amplification system for the target dsDNA fragment is shown in Table 2. The PCR reaction procedure was as follows: predenaturation at 95° C. for 10 min, denaturation at 95° C. for 15 s, annealing at 57° C. for 15 s, extension at 72° C. for 30 s (2 kb could be amplified in 1 minute), for 32 cycles, and finally, extension at 75° C. for 5 min. The fragment size was identified by 1.5% (w/v) agarose gel electrophoresis, and the amplified product was a correct single DNA fragment. The target fragment was recovered using a column recovery method with the Wizard SV Gel and PCR clean-up system kit from Promega.

TABLE 2
PCR Amplification System for Target dsDNA Fragment

| Component | Amount |
|---|---|
| 2xPhanta buffer (Nanjing Vazyme Biotech | 25 μL |
| Co., Ltd.) | |
| dNTP | 1 μL |
| DMSO | 5 μL |
| AMED16s-F (10 μM) | 2 μL |
| AMED16s-R (10 μM) | 2 μL |
| U32 genome | 1 μL |
| Phanta DNA polymerase (Nanjing Vazyme | 1 μL |
| Biotech Co., Ltd.) | |
| ddH₂O | up to 50 μL |

### 3. Cis-Cleavage Reaction Experiment:

TABLE 3
Cis-cleavage reaction system

| Component | Amount | |
|---|---|---|
| FnCas12a protein | 10 | μM |
| Target dsDNA | 100 | nM |
| crRNA | 50 | nM |
| 10x HOLMES buffer | 2 | μL |
| RRI (Takara) | 0.25 | μL |

| | |
|---|---|
| ddH₂O (RNase free) | Up to 20 μL |

TABLE 4
10x HOLMES buffer component

| | Concentration |
|---|---|
| Component | (mM or %) |
| Spermidine | 25 |
| Tris | 400 |
| MgCl₂ | 60 |
| DTT | 10 |
| Glycine | 400 |
| Triton X-100 | 0.01% |
| PEG20000 | 4% |
| pH | 8.4 |

crRNA Sequence:

5′-AAUUUCUACUCUUGUAGAUGCCAGGGACGAAGCGCAAGUGACGGAAU-3′ (SEQ ID NO.12), synthesized by Nanjingjinsirui Science & Technology Biology Corp., and purified by HPLC. The detection method was as follows: the reaction was carried out at 37° C. for 40 min, followed by inactivation at 85° C. for 5 min, and then a final concentration of 1×DNA loading was added. The entire reaction product was loaded, and 2% (w/v) agarose gel electrophoresis was performed at 140 V for 25 min. The gel was stained with EB for 30 min, and then imaged using a gel imager. The cis-cleavage products were DNA fragments of about 529 bp and 300 bp. Additionally, the Control experimental system did not include the addition of FnCas12a protein. The experimental results are shown in FIG. 2.

### 4. Trans-Cleavage Activity Detection Experiment

TABLE 5
Trans-cleavage Reaction System

| Component | Amount | |
|---|---|---|
| FnCas 12a protein | 5 | μM |
| target dsDNA | 30 | nM |
| crRNA | 50 | nM |
| 10x HOLMES buffer | 2 | μL |
| RRI (Takara) | 0.25 | μL |
| HOLMES-P (FQ-reporter) | 1 | μM |

| | |
|---|---|
| ddH₂O (RNase free) | Up to 20 μL |

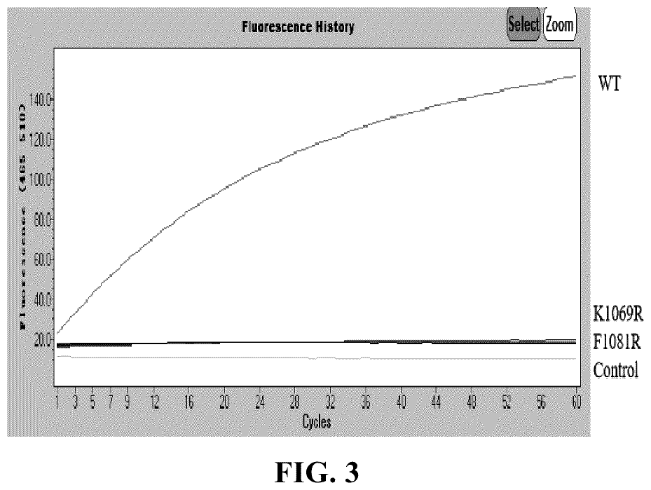

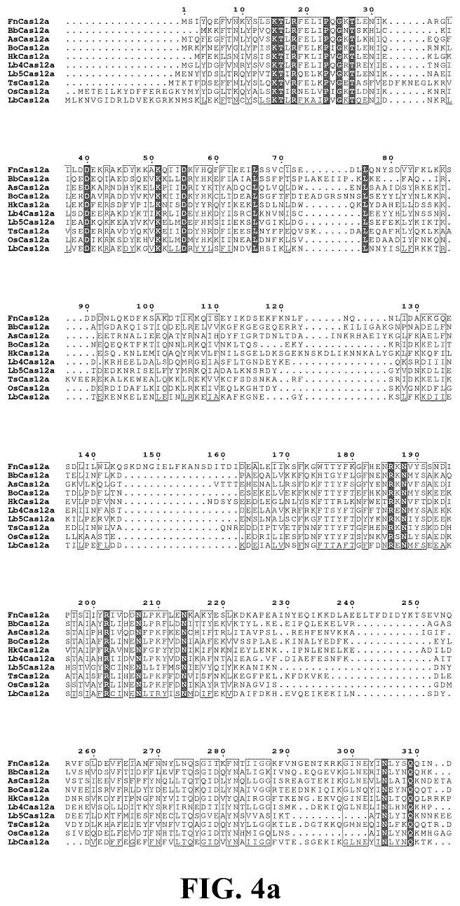

HOLMES-P (FQ-reporter), purchased from Anhui Tolo Port Biotechnology Co., Ltd., was a short single-stranded DNA probe (5′-TTTTTT-3′) modified with a FAM fluorescent luminescent group on one end and a fluorescence quenching group on the other end. When the short single-stranded DNA fragment was intact, the DNA probe would not emit fluorescence; only when the single-stranded DNA fragment was cleaved, and the quenching group was separated from the fluorescent group, could the fluorescence signal of the DNA probe be detected. Once the system was prepared, it was immediately placed in a real-time fluorescence quantitative PCR instrument to detect the fluorescence signal, subjected to incubation under the condition of 37° C., and the fluorescence signal was collected once every minute for a total of 30 times (for 60 min). The experimental results are shown in FIG. 3. Except for FnCas12a protein, all other components in the system were first prepared into a mixed system. Additionally, the Control experimental system did not include the addition of the target dsDNA.

### (II) Results and Discussion

The present invention analyzed the structure of FnCas12a. According to the results displayed by the crystal structure 6ilk, the FnCas12a amino acids that interact with DNA substrates comprise: K1069, F1081, F1010, V1285, N1288, etc. These amino acid sites may be related to the trans-cleavage activity. The present invention mutated these sites and determined the cis- and trans-cleavage activities of these proteins. Finally, two mutant proteins with cis-cleavage activity but no trans-cleavage activity were obtained. The mutations of these two proteins are the 1081st amino acid mutation from phenylalanine to arginine (F1081R) and the 1069th amino acid mutation from lysine to arginine (K1069R), respectively. The protein names are FnCas12a^{F1081R} and FnCas12a^{K1069R}, respectively. The purification results of wild-type protein (WT) and mutant proteins (F1081R and K1069R) are shown in FIG. 1. The results of cis-cleavage activity detection showed no significant difference in cis-cleavage activity between these two mutant proteins, FnCas12a^{F1081R} and FnCas12a^{K1069R}, and FnCas12a (FIG. 2). The results of trans-cleavage activity detection showed that the trans-cleavage activity of FnCas12a^{F1081R} and FnCas12a^{K1069R} was significantly reduced compared to the trans-cleavage activity of the wild-type FnCas12a protein (FIG. 3).

In summary, the present invention has discovered two mutant proteins of FnCas12a, with mutation sites at the 1081st amino acid changing from phenylalanine to arginine (F1081R) and the 1069th amino acid changing from lysine to arginine (K1069R), respectively. These two mutant proteins retain cis-cleavage activity while losing (or significantly reducing) the trans-cleavage activity of the original wild-type gene editing. Due to the fact that the wild-type Cas12a protein not only specifically cleaves target DNA but also has non-specific trans-cleavage activity towards single-stranded DNA, it can cause a certain degree of off-target effects during the gene editing process. In the present invention, by artificially modifying the wild-type gene editing protein, the trans-cleavage activity of Cas12a is removed (or reduced) while retaining its cis-cleavage activity. This modification overcomes the off-target issues caused by the trans-cleavage activity of the gene editing protein, thereby giving the Cas12a mutant protein a greater advantage in gene editing.

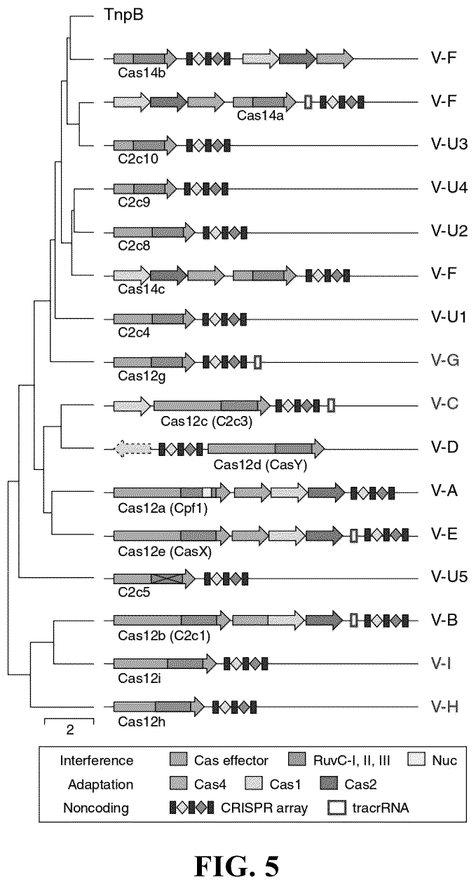

Additionally, the Class 2 Clustered Regularly Interspaced Short Palindromic Repeats (CRISPR)-Cas system is characterized by a single effector protein and can be further subdivided into types II, V, and VI, etc. The effector proteins of the Type V family exhibit diversity at the N-terminus, but retain a unified RuvC-like endonuclease domain at the C-terminus. The Type V system is further subdivided into many subtypes, including Type V-A to Type V-I, Type V-K, Type V-U, and CRISPR-Cas8φ (see FIG. 5). Cas12a (Type V-A), Cas12b (Type V-B), and Cas12e (Type V-E) all belong to the Type V system. They specifically recognize PAM rich in 5′-T after the effector protein binds to the gRNA to form a binary complex, and promote the unwinding of target DNA. Concurrently, the non-target strand (NTS) of the target sequence undergoes displacement, forming a so-called “R-loop” structure. The RuvC domain continuously cleaves the NTS and the target strand (TS) at a distance from the PAM, forming a staggered nick with 5, 7, or 10 NT strand 5′ overhangs. Cas12a, Cas12b, and Cas12e, all three proteins have a bilobed structure composed of an α-helical recognition (REC) lobe and a nuclease (NUC) lobe (see FIG. 7). The two lobes are connected by a bridge helix (BH) domain. The REC lobe contains two REC domains (REC1 and REC2), which primarily assist in modulating and stabilizing the hybridization between the crRNA target after forming the “R-loop” and the DNA. (Tong Baisong et al. The Versatile Type V CRISPR Effectors and Their Application Prospects [J]. Frontiers in Cell and Developmental Biology, 2021, 8:622103-622103.)

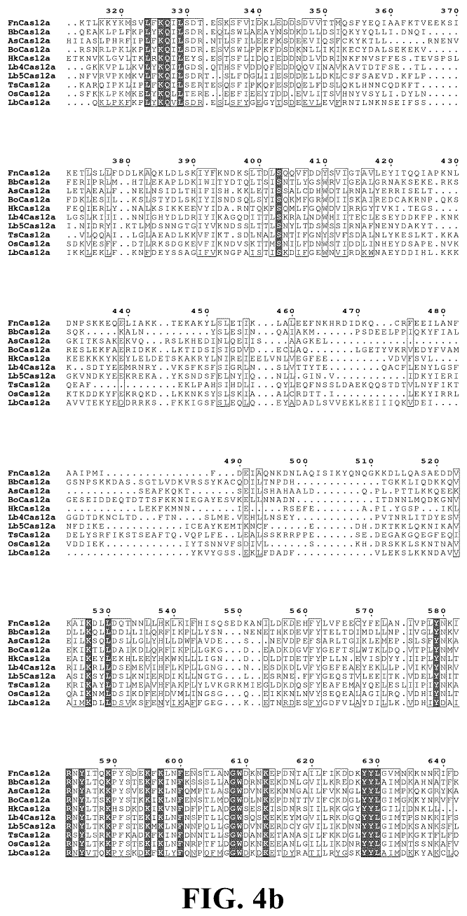

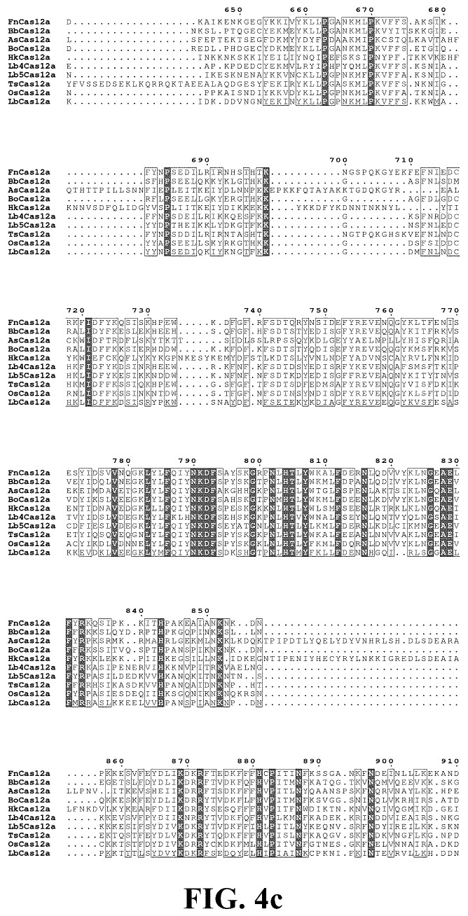

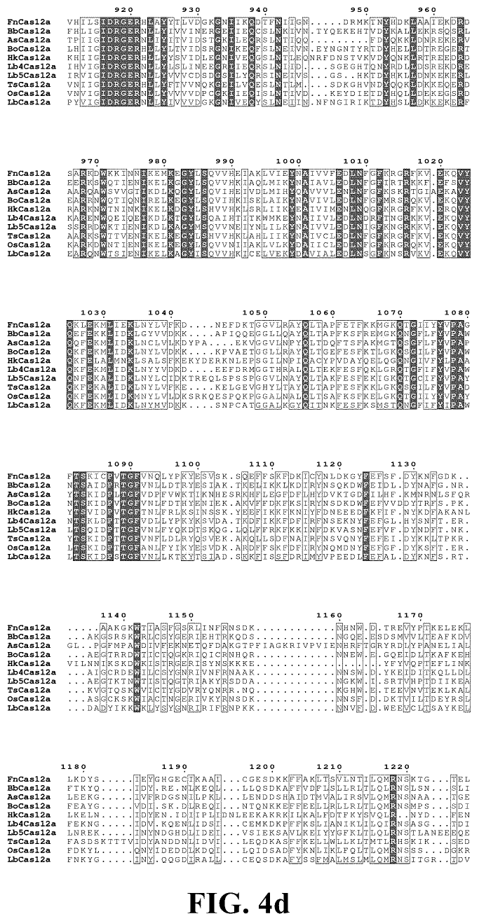

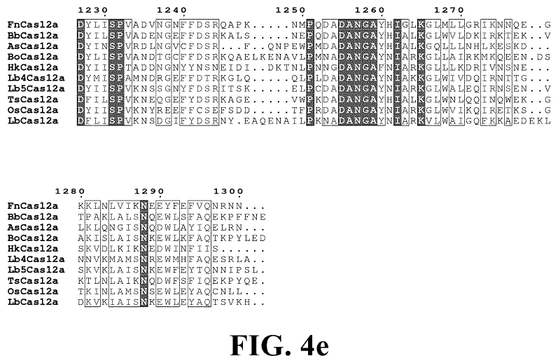

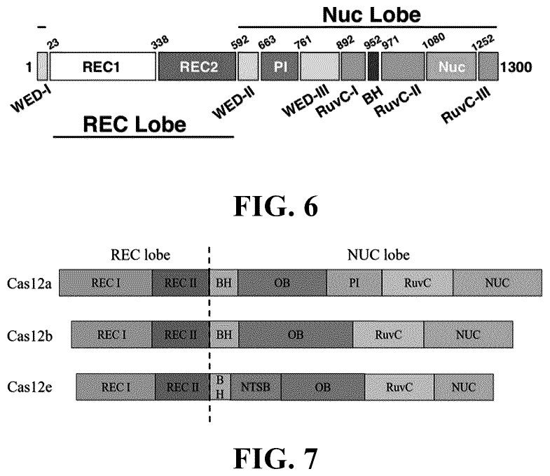

According to FIG. 6, the position 1069 of FnCas12a is located in the RuvC domain. Under the Type V system, each Cas protein has a RuvC domain (see FIG. 5) and corresponding sites. It can be anticipated that mutating the amino acid residues corresponding to position 1069 of FnCas12a in various Cas proteins under the Type V system will yield similar effects; especially for Cas12a, Cas12b, and Cas12e, which are structurally and functionally more similar and have higher homology, mutating the amino acid residues corresponding to position 1069 of FnCas12a will yield even more similar effects; and even more so for other sources of Cas12a (see FIG. 4a-4e) that are structurally and functionally more similar and have even higher homology, mutating the amino acid residues corresponding to position 1069 of FnCas12a (see Table II) will yield even more similar effects.

Similarly, according to FIG. 6, the position 1081 of FnCas12a is located at the junction of the RuvC domain and the NUC domain. Cas12a, Cas12b, and Cas12e all have RuvC domains and NUC domains (see FIG. 7). It can be anticipated that mutating the amino acid residues corresponding to position 1081 of FnCas12a in Cas12a, Cas12b, and Cas12e, which are structurally and functionally similar and have high homology, will yield similar effects; especially for other sources of Cas12a (see FIG. 4a-4e) that are structurally and functionally more similar and have higher homology, mutating the amino acid residues corresponding to position 1081 of FnCas12a (see Table II) will yield even more similar effects.

All literatures mentioned in the present invention are incorporated herein by reference, as though each one is individually incorporated by reference. In addition, it should be understood that, after reading the above teachings of the present invention, those skilled in the art can make various changes or modifications to the present invention, these equivalents also fall within the scope as defined in the appended claims of the present application.

1. A gene editing protein variant, which is an unnatural protein with cis-cleavage activity, and the variant has reduced trans-cleavage activity as compared to a wild-type gene editing protein thereof, and the variant undergoes mutation in the wild-type gene editing protein at one or more core amino acid sites selected from the group consisting of:
a phenylalanine (F) site corresponding to the 1081st position of FnCas12a; and/or
a lysine (K) site corresponding to the 1069th position of FnCas12a.

2. The gene editing protein variant of claim 1, wherein the phenylalanine (F) at the 1081st position of FnCas12a is mutated to one or more amino acids selected from the group consisting of: arginine (R), tyrosine (Y), tryptophan (W), glutamine (Q), asparagine (N), lysine (K), glutamic acid (E), aspartic acid (D), and a combination thereof.

3. The gene editing protein variant of claim 1, wherein the lysine (K) at the 1069th position of FnCas12a is mutated to one or more amino acids selected from the group consisting of: arginine (R), tyrosine (Y), glutamine (Q), asparagine (N), lysine (K), glutamic acid (E), aspartic acid (D), and a combination thereof.

4. The gene editing protein variant of claim 1, wherein the gene editing protein is a type-V CRISPR/Cas protein.

5. The gene editing protein variant of claim 1, wherein the gene editing protein is selected from the group consisting of: Cas12, Cas14, and a combination thereof.

6. The gene editing protein variant of claim 1, wherein the gene editing protein is selected from the group consisting of: Cas12a, Cas12b, Cas12e, and a combination thereof.

7. The gene editing protein variant of claim 1, wherein the gene editing protein is FnCas12a.

8. A polynucleotide encoding the variant of claim 1.

9. A vector comprising the polynucleotide of claim 8.

10. A host cell comprising a vector, or having the polynucleotide of claim 8 integrated into its genome;
wherein the vector comprises the polynucleotide of claim 8.

11. A method for preparing a gene editing protein variant, comprising the steps of:
(a) culturing the host cell of claim 10 under suitable conditions for expression, thereby expressing the gene editing protein variant; and
(b) isolating the gene editing protein variant.

12. An enzyme formulation comprising the gene editing protein variant of claim 1.

13. A gene editing system, comprising:
the gene editing protein variant of claim 1, or a coding gene thereof, or an expression vector thereof; and
a gRNA or an expression vector thereof, and/or an oligonucleotide or nucleic acid fragment or plasmid thereof used for target site break repair.

14. A gene editing reagent comprising the gene editing protein variant of claim 1.

15. A composition comprising:
the gene editing protein variant of claim 1, or a system, or a gene editing reagent; and
a pharmaceutically acceptable carrier;
wherein the system comprises:
the gene editing protein variant of claim 1, or a coding gene thereof, or an expression vector thereof; and
a gRNA or an expression vector thereof, and/or an oligonucleotide or nucleic acid fragment or plasmid thereof used for target site break repair;
wherein the gene editing reagent comprises the gene editing protein variant of claim 1.

16. A product combination comprising:
the gene editing protein variant of claim 1, or a system, or a gene editing reagent;
wherein the system comprises:
the gene editing protein variant of claim 1, or a coding gene thereof, or an expression vector thereof; and
a gRNA or an expression vector thereof, and/or an oligonucleotide or nucleic acid fragment or plasmid thereof used for target site break repair;
wherein the gene editing reagent comprises the gene editing protein variant of claim 1.

17. A kit comprising: the gene editing protein variant of claim 1, or an enzyme formulation, or a gene editing system, or a gene editing reagent, or a composition, or a product combination;
wherein the enzyme formulation comprises the gene editing protein variant of claim 1;
wherein the gene editing system comprises:
the gene editing protein variant of claim 1, or a coding gene thereof, or an expression vector thereof; and
a gRNA or an expression vector thereof, and/or an oligonucleotide or nucleic acid fragment or plasmid thereof used for target site break repair;
wherein the gene editing reagent comprises the gene editing protein variant of claim 1;
wherein the composition comprises:
the gene editing protein variant of claim 1, or the gene editing system, or the gene editing reagent; and
a pharmaceutically acceptable carrier;
wherein the product combination comprises:
the gene editing protein variant of claim 1, or the gene editing system, or the gene editing reagent.

18. A medical kit comprising:
a first container, and the gene editing protein variant of claim 1, or an enzyme formulation, or a gene editing system, or a gene editing reagent, or a composition, or a product combination, or a drug comprising the gene editing protein variant of claim 1, or the enzyme formulation, or the gene editing system, or the gene editing reagent, or the composition, or the product combination, located in the first container;
wherein the enzyme formulation comprises the gene editing protein variant of claim 1;
wherein the gene editing system comprises:
the gene editing protein variant of claim 1, or a coding gene thereof, or an expression vector thereof; and
a gRNA or an expression vector thereof, and/or an oligonucleotide or nucleic acid fragment or plasmid thereof used for target site break repair;
wherein the gene editing reagent comprises the gene editing protein variant of claim 1;
wherein the composition comprises:
the gene editing protein variant of claim 1, or the gene editing system, or the gene editing reagent; and
a pharmaceutically acceptable carrier;
wherein the product combination comprises:
the gene editing protein variant of claim 1, or the gene editing system, or the gene editing reagent.

19. A medical kit comprising:
(a1) a first container, and the gene editing protein variant of claim 1, or a coding gene thereof, or an expression vector thereof, or a drug comprising the gene editing protein variant of claim 1, or a coding gene thereof, or an expression vector thereof, located in the first container;
(b1) a second container, and a gRNA or an expression vector thereof, or a drug comprising a gRNA or an expression vector thereof, located in the second container.

20. (canceled)

21. A method for reducing a gene editing off-target rate, comprising the step of:
in the presence of the gene editing protein variant of claim 1, or an enzyme formulation, or a gene editing system, or a gene editing reagent, or a composition, or a product combination, or a medical kit, performing gene editing on a cell, thereby reducing the gene editing off-target rate;
wherein the enzyme formulation comprises the gene editing protein variant of claim 1;
wherein the gene editing system comprises:
the gene editing protein variant of claim 1, or a coding gene thereof, or an expression vector thereof; and
a gRNA or an expression vector thereof, and/or an oligonucleotide or nucleic acid fragment or plasmid thereof used for target site break repair;
wherein the gene editing reagent comprises the gene editing protein variant of claim 1;
wherein the composition comprises:
the gene editing protein variant of claim 1, or the gene editing system, or the gene editing reagent; and
a pharmaceutically acceptable carrier;
wherein the product combination comprises:
the gene editing protein variant of claim 1, or the gene editing system, or the gene editing reagent;
wherein the medical kit comprises:
a first container, and the gene editing protein variant of claim 1, or the enzyme formulation, or the gene editing system, or the gene editing reagent, or the composition, or the product combination, or a drug comprising the gene editing protein variant of claim 1, or the enzyme formulation, or the gene editing system, or the gene editing reagent, or the composition, or the product combination, located in the first container;
or the medical kit comprises:
(a1) a first container, and the gene editing protein variant of claim 1, or a coding gene thereof, or an expression vector thereof, or a drug comprising the gene editing protein variant of claim 1, or a coding gene thereof, or an expression vector thereof, located in the first container:
(b1) a second container, and a gRNA or an expression vector thereof, or a drug comprising a gRNA or an expression vector thereof, located in the second container.
